(19) United States
(12) Patent Application Publication
(10) Pub. No.: US 2024/0124409 A1
(43) Pub. Date: Apr. 18, 2024

(54) GUAIANOLIDE SESQUITERPENE LACTONE DERIVATIVES AND PHARMACEUTICAL USE THEREOF

(71) Applicant: NANJING UNIVERSITY OF CHINESE MEDICINE, Jiangsu (CN)

(72) Inventors: Lihong HU, Jiangsu (CN); Jian LIU, Jiangsu (CN); Qi LV, Jiangsu (CN); Yang HU, Jiangsu (CN); Dong DONG, Jiangsu (CN); Ping WANG, Jiangsu (CN); Meng YANG, Jiangsu (CN)

(73) Assignee: NANJING UNIVERSITY OF CHINESE MEDICINE, Jiangsu (CN)

(21) Appl. No.: 18/519,062

(22) Filed: Nov. 26, 2023

Related U.S. Application Data
(63) Continuation of application No. PCT/CN22/95243, filed on May 26, 2022.

(30) Foreign Application Priority Data
May 28, 2021 (CN) 202110588960.4
May 25, 2022 (CN) 202210573181.1

(51) Int. Cl. C07D 307/93; A61P 1/16; A61P 11/00; A61P 13/12; A61P 19/02; A61P 19/06; A61P 29/00; A61P 31/00; A61P 35/00

(52) U.S. Cl. CPC C07D 307/93; A61P 1/16; A61P 11/00; A61P 13/12; A61P 19/02; A61P 19/06; A61P 29/00; A61P 31/00; A61P 35/00

(57) ABSTRACT

The present application discloses a class of guaianolide sesquiterpene lactone derivatives and pharmaceutical use thereof. The guaianolide sesquiterpene lactone derivative or a pharmaceutically acceptable salt thereof is shown as general formula I. In the present application, a class of novel guaianolide sesquiterpene lactone derivatives are found by structural optimization with abundant natural ingredients such as parthenolide and dehydrocostus lactone as raw materials, which derivatives have good inhibitory activity on the activation of the NLRP3 inflammasome, and the chemical stability, water solubility and oral bioavailability of which are significantly improved, and it is verified by experiments that the derivatives have inhibitory effects on the activity of the NLRP3 inflammasome and have potential pharmaceutical applications.

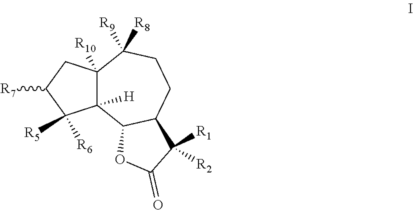

### CROSS-REFERENCE TO RELATED APPLICATION

This application is a continuation in-part of international application of PCT application serial no. PCT/CN2022/095243, filed on May 26, 2022, which claims the priority benefit of China application no. 202110588960.4, filed on May 28, 2021 and China application no.202210573181.1 and May 25, 2022. The entirety of each of the above-mentioned patent applications is hereby incorporated by reference herein and made a part of this specification.

### BACKGROUND OF THE INVENTION

### 1. Technical Field

The present application relates to sesquiterpene lactone derivatives and use thereof, specifically to a class of guaianolide sesquiterpene lactone derivatives and pharmaceutical use thereof.

### 2. Background Art

Inflammasome is a protein complex in innate immune cells such as macrophages, monocytes and dendritic cells, which can recognize PAMPs (pathogen-associated molecular patterns) or/and DAMPs (damage-associated molecular patterns) [Front Immunol, 2019, 10: 2538.]. Different types of inflammasomes, such as NLRP1, NLRP3, NLRC4, Pyrin, NLRP6 and AIM2, etc., can mediate inflammatory responses, promote the release of inflammatory cytokines, and transmit signals to the immune system, which are the initiators of inflammation and the bridges between natural immunity and acquired immunity [Cell, 2016, 165:792-800.]. Different from other types of inflammasomes that recognize specific DAMPs or PAMPs, the NLRP3 inflammasome can broadly recognize DAMPs and PAMPs from different sources. Therefore, the study of the NLRP3 inflammasome is of great interests in different fields. It is also the most deeply studied inflammasome at present, and has been proved to be involved in the occurrence and development of a variety of chronic inflammatory diseases [Nat Rev Drug Discov, 2018, 17:588-606.].

The NLRP3 inflammasome consists of the receptor protein NLRP3, the regulatory protein ASC and the effector protein pro-Caspase-1 [Immunol Rev, 2015, 265: 35-52.]. The activation of the NLRP3 inflammasome is divided into two steps: step 1: the TLR4 receptor recognizes the first signal, such as PAMPs, DAMPs or exogenous stress molecules, etc., and up-regulates the protein expression of NLRP3, pro-IL-1β and pro-IL-18 by activating the NF-κB pathway; and step 2: the receptor protein NLRP3 recognizes the second signal, such as PAMPs, DAMPs or intracellular stress molecules, etc., activates pro-Caspase-1 by combining with the adaptor protein ASC, and then cleaves and activates pro-IL-1β and pro-IL-18 to promote the maturation and secretion of IL-1β and IL-18 [Int J Mol Sci, 2019, 20: 3328.]. IL-1β will further activate the NF-KB signaling pathway through the autocrine and paracrine pathways, promote the secretion of cytokines such as IL-1β, TNF-α, IL-6 and IL-8, etc., and trigger inflammatory cascade reactions, leading to the occurrence and development of chronic diseases [Front Immunol, 2019, 10: 276.].

The overactivation of the NLRP3 inflammasome is closely related to the occurrence and development of many diseases, including immune diseases, autoimmune diseases, malignant tumors, skin diseases, cardiovascular diseases, liver-associated diseases, kidney system-associated diseases, gastrointestinal tract-associated diseases, central nervous system diseases, metabolic diseases, endocrine-associated diseases, respiratory diseases, lymphatic system diseases, inflammation, infectious diseases, ocular diseases, psychological disorders and pain, etc. [Nat Med, 2015, 21: 248-255; J Clin Invest, 2020, 130: 1961-1976; Cell Metab, 2020, 31: 580-591; Circ Res, 2018, 122: 1722-1740; J Hepatol, 2017, 66: 1037-1046; Ageing Res Rev, 2020, 64: 101192; Autophagy, 2019, 15: 1860-1881; Brain, 2020, 143: 1414-1430; Mucosal Immunol, 2019, 12: 1150-1163; J Clin Invest, 2018, 128: 1793-1806; Immunology, 2020, 160: 78-89; J Inflamm (Lond), 2015, 12: 41; Nat Commun, 2020, 11: 4243; Front Immunol, 2020, 11: 570251; Biochem Biophys Res Commun, 2016, 477: 329-335; Pharmaceutics, 2020, 12: 867; Arthritis Rheumatol, 2020, 72:1192-1202; Food Chem Toxicol, 2020, 144: 111588; EMBO Rep, 2020, 21: e49666; Int Immunopharmacol, 2020, 81: 106257; Cells, 2019, 8: 1389; Cell Prolif, 2021, 54: e12973.]. Therefore, the above disease can be prevented and/or treated by inhibiting the activation of the NLRP3 inflammasome.

The structural formula of arglabin is as follows:

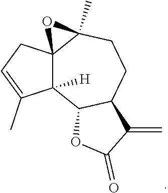

Abderrazak A's group found that arglabin, a guaianolide sesquiterpene lactone, exhibited extremely potent inhibitory activity against the NLRP3 inflammasome activation (EC₅₀=10 nM). Arglabin can alleviate the NLRP3-related inflammatory diseases, protect pancreatic β-cells and prevent type 2 diabetes [Circulation, 2015, 131: 1061-1070; J Pharmacol Exp Ther, 2016, 357: 487-494.]. Arglabin is derived from the plant Wormwood (Artemisia) in Kazakhstan, with a low content of about 0.27% [J Nat Prop, 1999, 62: 1068-1071.]; its water-solubility was only 7.9 μg/mL; and it has a poor chemical stability in gastric juice environments, with the degradation ratio of 50% within 8 h, and the oral bioavailability of only 5%, which shortcomings in druggability limit its clinical application. Therefore, the chemical stability, water solubility, oral bioavailability and resource supply economy of such compounds need to be further improved.

### SUMMARY OF THE INVENTION

Purpose of the present application: The purpose of the present application is to provide a guaianolide sesquiterpene lactone derivative to improve the chemical stability, water solubility and oral bioavailability of such compounds. Another purpose of the present application is to provide use of such compounds in preparation of a medicine for treating NLRP3 inflammasome-associated diseases.

Technical scheme: Guaianolide sesquiterpene lactone derivatives of general formula I, or a pharmaceutically acceptable salt thereof, wherein

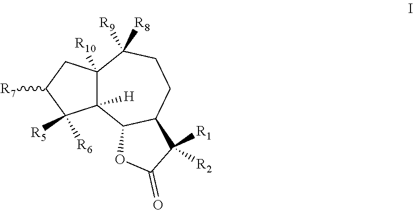

R¹ and R² together form a double bond; or R¹ is hydrogen or deuterium, R² is

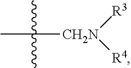

wherein R³ and R⁴ are C₁-C₆ alkyl groups respectively, or R³, R⁴ and a N atom form a 5-6 membered ring structure, on which the ring structure is selected from pyrrole, piperidine, piperazine, and morpholine;
R⁵ is methyl; and R⁶ is a hydroxyl, C₁-C₆ alkoxy, C₁-C₆ ester group, halogen or forms a double bond with an adjacent carbon atom;
R⁷ is hydrogen or hydroxyl; and
R⁸ is methyl, and R′ is connected with R¹⁰ directly to form cyclopropane.

Further preferably, the guaianolide sesquiterpene lactone derivatives or a pharmaceutically acceptable salt thereof are selected from the following compounds:

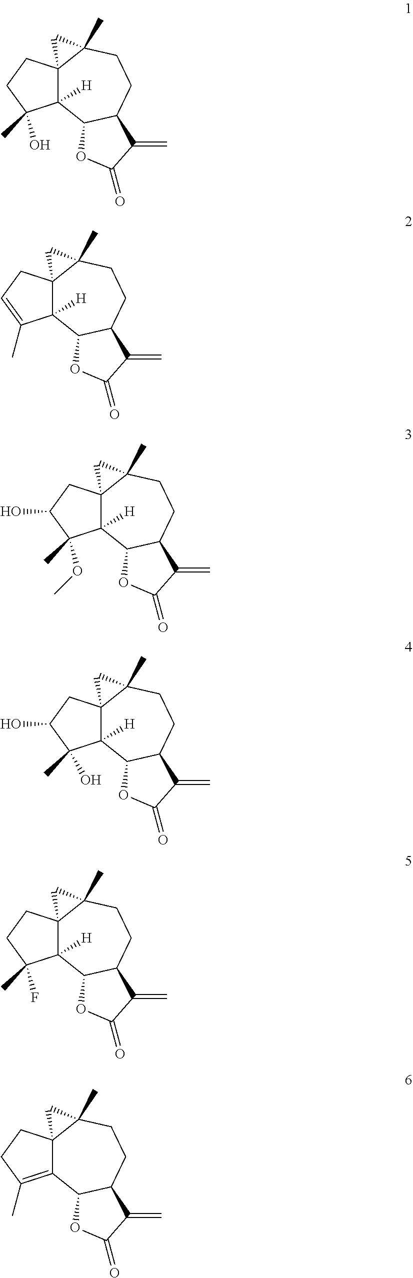

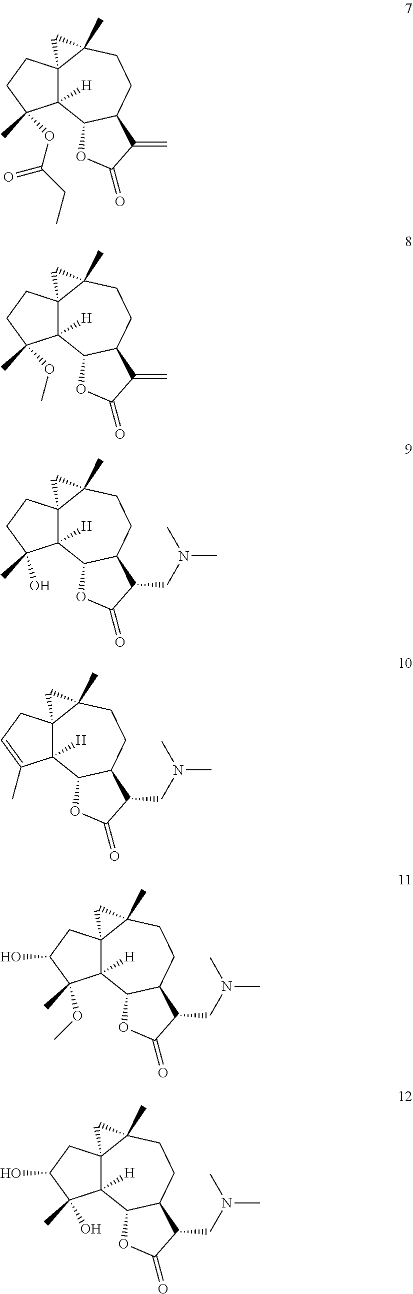

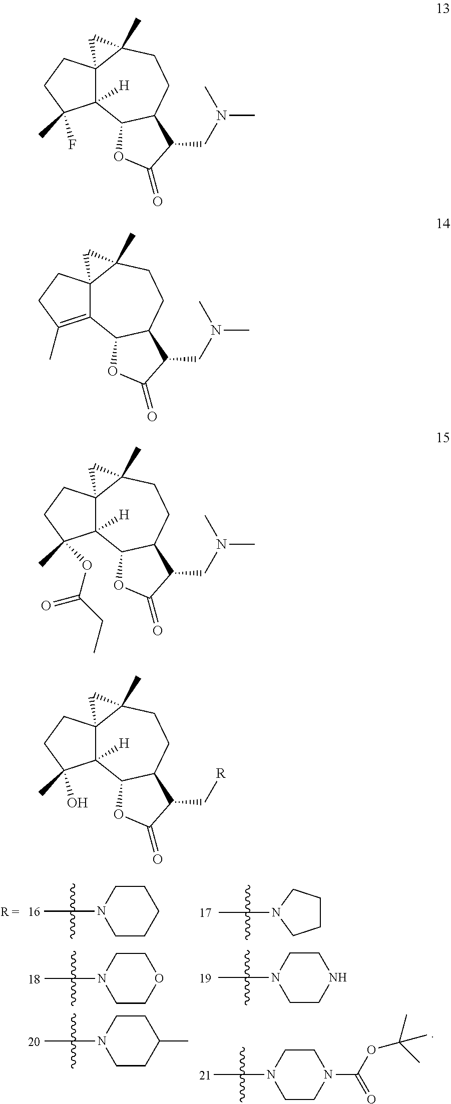

Preferably, the guaianolide sesquiterpene lactone derivatives or a pharmaceutically acceptable salt thereof refer to a pharmaceutically acceptable salt formed with an inorganic or organic acid, including a hydrochloride, a sulfate, a phosphate, a maleate, a fumarate, and a citrate, etc.

Further, the pharmaceutically acceptable salt is selected from:

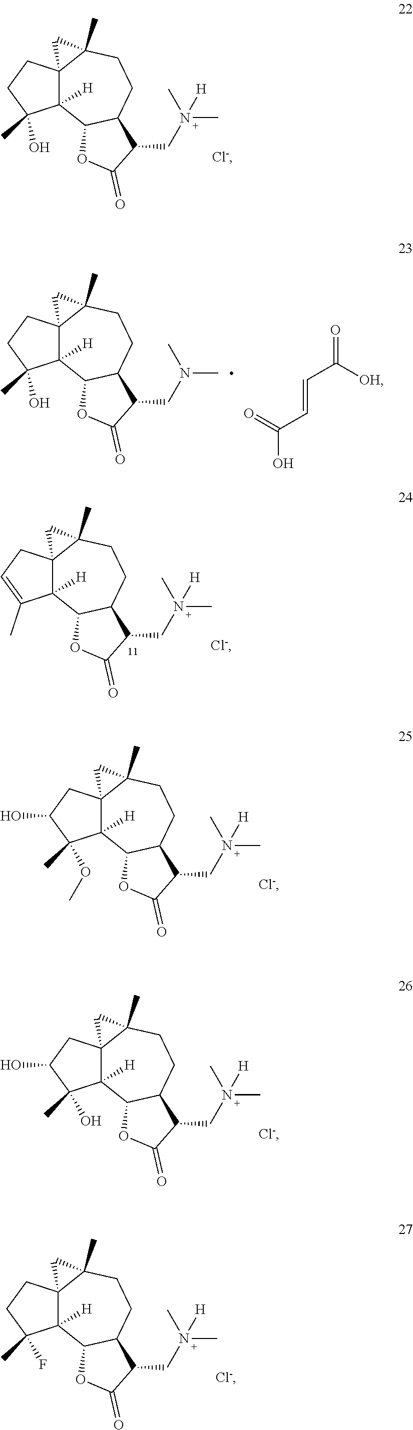

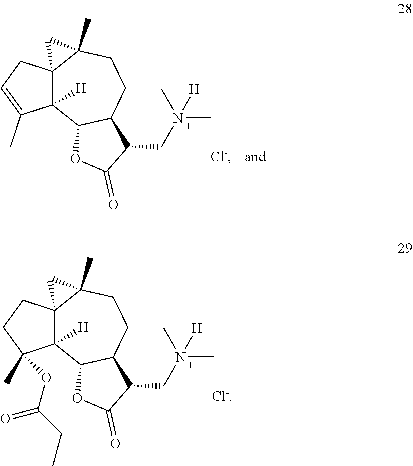

This application further discloses a method for preparation of the guaianolide sesquiterpene lactone derivatives above.

This application further discloses a pharmaceutical composition comprising a therapeutically effective amount of one or more selected from the guaianolide sesquiterpene lactone derivatives above or a pharmaceutically acceptable salt thereof as active ingredients. The pharmaceutical composition further comprises a pharmaceutically acceptable carrier, adjuvant or auxiliary material.

This application further discloses use of the guaianolide sesquiterpene lactone derivatives or a pharmaceutically acceptable salt thereof and the pharmaceutical composition above in preparation of a medicine for preventing or treating NLRP3 inflammasome-associated diseases.

This application further discloses use of the guaianolide sesquiterpene lactone derivatives above or a pharmaceutically acceptable salt thereof in combination with other pharmaceutically acceptable therapeutic agents, especially other NLRP3 inflammasome inhibitors, in preparation of a medicine for preventing or treating NLRP3 inflammasome-associated diseases.

The present application provides a method for preventing or treating NLRP3 inflammasome-associated diseases, including administering a therapeutically effective amount of one or more selected from the guaianolide sesquiterpene lactone derivatives according to the present application or a pharmaceutically acceptable salt thereof, or the pharmaceutical composition according to the present application comprising a therapeutically effective amount of one or more selected from the guaianolide sesquiterpene lactone derivatives according to the present application or a pharmaceutically acceptable salt thereof as active ingredients to a patient in need thereof.

The NLRP3 inflammasome-associated diseases include: immune diseases, autoimmune diseases, malignant tumors, skin diseases, cardiovascular diseases, liver-associated diseases, kidney system-associated diseases, gastrointestinal tract-associated diseases, central nervous system diseases, metabolic diseases, endocrine-associated diseases, respiratory diseases, lymphatic system diseases, inflammation, infectious diseases, ocular diseases, psychological disorders and pain, etc.

Specifically, the diseases include: (1) Cryopyrin-associated periodic syndromes (CAPS): Muckle-Wells syndromes (MWS), familial cold autoinflammatory syndromes (FCAS) and neonatal-onset multisystem inflammatory diseases NOMID; (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis (MS), type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury diseases.

Beneficial effect: Compared with the existing technology, in the present application, the guaianolide sesquiterpene lactone derivatives of general formula I are synthesized with high stereoselectivity by specific structural modification with abundant natural ingredients such as parthenolide and dehydrocostus lactone as raw materials, and the cyclopropane configurations of the obtained compounds are all a configurations. The experimental results show that the inhibitory activity of these compounds on the activation of the NLRP3 inflammasome is maintained, and their chemical stability, water solubility and oral bioavailability are significantly improved, so they have better development and application prospects.

### BRIEF DESCRIPTION OF THE DRAWINGS

FIG. 1 shows the change of the concentrations of Compound 1 and Compound 23 in the HEPES7.4 solution with time; and

FIG. 2 shows the change of the concentrations of Compound 1 and Compound 23 in mouse plasma with time.

### DETAILED DESCRIPTION OF THE EMBODIMENTS

This application will be described in detail in combination with specific examples.

### Example 1: Preparation of Parthenolide and Dehydrocostus Lactone

Preparation of parthenolide. 5 kg of the dried root bark of Magnolia delavayi was crushed

into coarse powder, soaked in 10×95% ethanol for 12 h, heated, refluxed and extracted twice with 2 h each time, and filtered. The filtrates were combined, concentrated under reduced pressure and dried to obtain the coarse extract of Magnolia delavayi. It was further refined by silica gel column chromatography, eluting with petroleum ether-ethyl acetate gradient to collect fractions rich in parthenolide and costus lactone in stages, which were combined, concentrated, and recrystallized to obtain parthenolide, with a preparation yield of 4.0% and a purity of 96.3%. ¹H NMR (500 MHz, CDCl₃): δ 6.31 (d, J=2.9 Hz, 1H), 5.62 (d, J=3.4 Hz, 1H), 5.20 (d, J=11.8 Hz, 2H), 3.85 (t, J=8.6 Hz, 1H), 2.78 (d, J=8.9 Hz, 1H), 2.45-2.32(m, 2H), 2.22-2.10 (m, 4H), 1.70 (s, 3H), 1.69-1.66 (m, 1H), 1.29 (s, 3H), 1.27-1.18 (m, 1H). ESI-MS (m/z): [M+H]⁺=249.1 (calcd: 249.1).

Preparation of dehydrocostus lactone. 5 kg of the medicine Saussurea costus was crushed into coarse powder, soaked in 8× petroleum ether for 12 h, heated, refluxed and extracted twice with 2 h each time, and filtered. The filtrates were combined, concentrated under reduced pressure and dried to obtain the coarse extract of Saussurea costus. It was further refined by silica gel column chromatography, eluting with petroleum ether-ethyl acetate gradient to collect fractions rich in dehydrocostus lactone in stages, which were combined, concentrated, and recrystallized to obtain dehydrocostus lactone, with a preparation yield of 1.0% and a purity of 96.8%. ¹H NMR (500 MHz): δ 6.22 (d, J=3.3 Hz, 1H), 5.49 (d, J=3.2 Hz, 1H), 5.27 (d, J=2.0 Hz, 1H), 5.07 (d, J=2.0 Hz, 1H), 4.90 (s, 1H), 4.82 (s, 1H), 3.97-3.94 (m, 1H), 2.95-2.88 (m, 2H), 2.87 (dd, J=9.3, 3.0 Hz, 1H), 2.24-2.22 (m, 1H), 2.16-2.13 (m, 1H), 1.99-1.96 (m, 2H), 1.88-1.86 (m, 2H), 1.42-1.40 (m, 2H). ESI-MS (m/z): [M+H]⁺=231.1 (calcd: 231.1).

### Example 2: Synthesis of Compounds 1-8

Although arglabin has a good inhibitory effect on the activation of the NLRP3 inflammasome, the epoxy ring in its structure can be hydrolyzed to open the ring under acidic conditions, and the test found that its degradation ratio reached 50% within 8 h. Therefore, we replaced the epoxy ring in arglabin with cyclopropane, hoping to improve its chemical stability while maintaining its activity.

### Synthesis of Compound 1

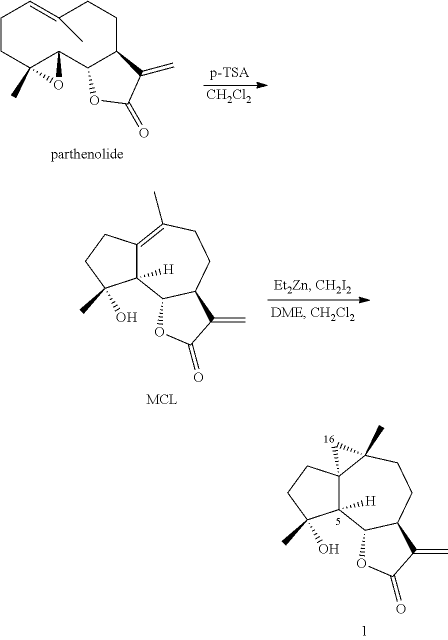

Dichloromethane (50 mL), p-toluenesulfonic acid (125 mg, 0.73 mmol) and parthenolide (5 g, 20.16 mmol) were added into a 150 mL round-bottomed flask sequentially, and stirred at room temperature until the completion of the reaction was detected by TLC. The reaction solution was washed with water (10 mL×3) and saturated sodium chloride solution (10 mL×3) sequentially, dried with anhydrous sodium sulfate, and then concentrated under reduced pressure, and purified by silica gel column chromatography to obtain the micheliolide MCL with a yield of 90%. ¹H NMR (500 MHz, CDCl₃): δ 6.21 (d, J=3.5 Hz, 1H), 5.51 (d, J=3.0 Hz, 1H), 3.81 (t, J=10.5 Hz, 1H), 2.73 (d, J=10.5 Hz, 1H), 2.68-2.64 (m, 2H), 2.42-2.37 (m, 1H), 2.26-2.16 (m, 3H), 2.11-2.08 (m, 1H), 1.83-1.75 (m, 2H), 1.69 (s, 3H), 1.31 (s, 3H), 1.27-1.25 (m, 1H). ESI-MS (m/z): [M+Na]⁺=271.1 (calcd: 271.1).

Under the condition of an ice bath and nitrogen protection, ethylene glycol dimethyl ether

(1.67 mL, 21.26 mmol) was added to anhydrous dichloromethane (67 mL), into which was added 13.3 mL of a diethyl zinc solution (1 M n-hexane solution) after stirring uniformly, and slowly dropped diiodomethane (2.67 mL, 3.11 mmol), and stirred for 10 min to prepare a cyclopropanation reagent. Micheliolide MCL (300 mg, 1.21 mmol) and anhydrous dichloromethane (5 mL) were added into another round-bottomed flask, dissolved by stirring, and then protected by nitrogen, and placed on an ice bath. The cyclopropanation reagent above was added to the substrate solution dropwise. After the dropwise addition was completed, the reaction was continued for 1 h, and the reaction was transferred to room temperature for overnight reaction. The reaction solution was quenched with saturated ammonium chloride, filtered, and then washed with water (10 mL×3) and saturated sodium chloride solution (10 mL×3) sequentially, dried with anhydrous sodium sulfate, and then concentrated under reduced pressure, and purified by silica gel column chromatography to obtain Compound 1 with a yield of 75%. ¹H NMR (500 MHz, CDCl₃): δ 6.14 (d, J=3.5 Hz, 1H), 5.45 (d, J=3.0 Hz, 1H), 3.82 (t, J=10.5 Hz, 1H), 2.52-2.48 (m, 1H), 2.26-2.21 (m, 1H), 2.09-1.99 (m, 2H), 1.93-1.89 (m, 1H), 1.88-1.86 (m, 1H, H₅), 1.85-1.83 (m, 1H), 1.55 (s, 3H), 1.52-1.44 (m, 2H), 1.26-1.14 (m, 2H), 1.11 (s, 3H), 0.81 (d, J=4.0 Hz, 1H, H₁₆ₐ), 0.54 (d, J=4.0 Hz, 1H, H_{16b}). The ROESY spectrum shows that there is a signal correlation between H₅ and H₁₆ₐ, which confirms that the cyclopropane is in a configuration. ESI-MS (m/z): [M+Na]⁺=285.2 (calcd: 285.2).

### Preparation of Compound 2

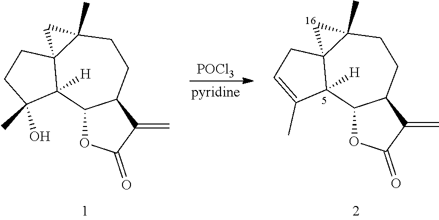

Compound 1 (200 mg, 0.81 mmol) and anhydrous pyridine (10 mL) were added into a round-bottomed flask sequentially, protected by nitrogen, and dissolved on an ice water bath, into which was added phosphorus oxychloride (1242 mg, 8.10 mmol) dropwise, and then transferred to room temperature for reaction for 2 h. After the dropwise addition was completed, the reaction was transferred to room temperature for 2 h reaction. The reaction solution was poured into ice water and extracted with ethyl acetate (10 mL×3). The organic layer was washed with a saturated copper sulfate solution (10 mL×6), water (10 mL×3) and saturated sodium chloride solution (10 mL×3) sequentially, dried with anhydrous sodium sulfate, and then concentrated under reduced pressure, and purified by silica gel column chromatography to obtain Compound 2 with a yield of 65%. ¹H NMR (500 MHz, CDCl₃): δ 6.14 (d, J=3.5 Hz, 1H), 5.59-5.57 (m, 1H), 5.44 (d, J=3.0 Hz, 1H), 3.90 (t, J=10.5 Hz, 1H), 2.74-2.71 (m, 1H), 2.51-2.46 (m, 1H), 2.32-2.28 (m, 2H), 2.04-2.00 (m, 1H), 1.97 (s, 3H), 1.81-1.77 (m, 1H, H₅), 1.54-1.46 (m, 1H), 1.18-1.14 (m, 1H), 1.13 (s, 3H), 0.61 (d, J=4.0 Hz, 1H, H₁₆ₐ), 0.50 (d, J=4.0 Hz, 1H, H_{16b}). The ROESY spectrum shows that there is a signal correlation between H₅ and H₁₆ₐ, which confirms that the cyclopropane is in a configuration. ESI-MS (m/z): [M+Na]⁺=267.1 (calcd: 267.2).

### Preparation of Compound 3

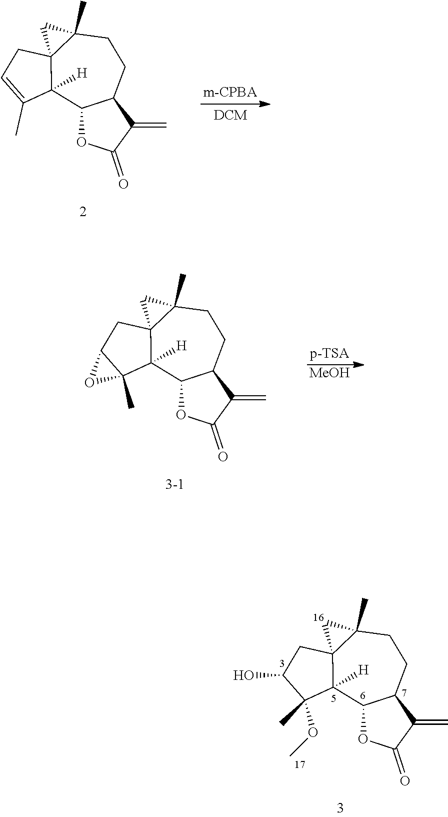

M-chloroperoxybenzoic acid (267.5 mg, 1.55 mmol) and anhydrous dichloromethane (20 mL) were added into a round-bottomed flask sequentially, into which the solution of Compound 2 (248 mg, 1.00 mmol) in dichloromethane (5 mL) was slowly added, and then stirred overnight. The reaction was quenched with a saturated sodium thiosulfate solution, washed with saturated sodium bicarbonate (10 mL×3), water (10 mL×3) and saturated sodium chloride solution (10 mL×3) sequentially, dried with anhydrous sodium sulfate, and then concentrated under reduced pressure, and purified by silica gel column chromatography to obtain Compound 3-1 with a yield of 83%. ¹H NMR (500 MHz, CDCl₃): δ 6.18 (d, J=3.5 Hz, 1H), 5.48 (d, J=3.0 Hz, 1H), 3.79 (t, J=10.5 Hz, 1H, H₆), 2.53-2.48 (m, 1H), 2.28-2.24 (m, 1H, H₃), 2.16-2.10 (m, 2H), 2.04-2.01 (m, 1H, H₅), 1.73 (s, 3H), 1.56-1.45 (m, 3H), 1.12-1.09 (m, 1H), 1.08 (s, 3H), 0.57 (d, J=4.0 Hz, 1H, H₁₆ₐ), 0.49 (d, J=4.0 Hz, 1H, H_{16b}). The ROESY spectrum shows that there is a signal correlation between H₅ and H₁₆ₐ, which confirms that the cyclopropane is in a configuration, and the epoxypropane is in a configuration. ESI-MS (m/z): [M+Na]⁺=283.1 (calcd: 283.1).

Intermediate 3-1 (260 mg, 1.00 mmol), methanol (10 mL) and p-toluenesulfonic acid (172 mg, 1.00 mmol) were added to a round-bottomed flask sequentially, and stirred overnight. After concentration, the reaction solution was extracted with ethyl acetate (10 mL×3). The organic layer was washed with saturated sodium bicarbonate (10 mL×3), water (10 mL×3) and saturated sodium chloride solution (10 mL×3) sequentially, dried with anhydrous sodium sulfate, and then concentrated under reduced pressure, and purified by silica gel column chromatography to obtain Compound 3 with a yield of 85%. ¹H NMR (500 MHz, CDCl₃): δ 6.15 (d, J=3.5 Hz, 1H), 5.44 (d, J=3.0 Hz, 1H), 4.25 (t, J=10.5 Hz, 1H, H₆), 3.64-3.62 (m, 1H, H₃), 3.41 (s, 3H, H₁₇), 2.55-2.48 (m, 1H, H₇), 2.41 (s, 1H), 2.22-2.18 (m, 1H), 2.08 (d, J=11.0 Hz, 1H), 2.06-2.01 (m, 1H), 1.98-1.94 (m, 1H), 1.80-1.75 (m, 1H, H₅), 1.61-1.55 (m, 1H), 1.49 (s, 3H), 1.47-1.42 (m, 1H), 1.10 (s, 3H), 0.57 (d, J=4.0 Hz, 1H, H₁₆ₐ), 0.49 (d, J=4.0 Hz, 1H, H_{16b}). ROESY shows that there are signal correlations between H₃ and H₆, H₅ and H₁₆ₐ, and H₇ and H₁₇, which confirms that the cyclopropane is in a configuration, and 3-OH and 4-OMe are in a configuration. ESI-MS (m/z): [M+Na]⁺=315.1 (calcd: 315.1).

### Preparation of Compound 4

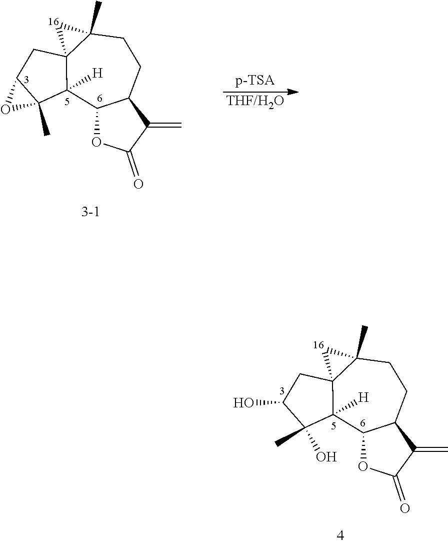

Tetrahydrofuran (5 mL) and water (1 mL) were added into a round-bottomed flask, into which were added p-toluenesulfonic acid (272 mg, 1.00 mmol) and Intermediate 3-1 (260 mg, 1.00 mmol), and stirred overnight. After concentration, the reaction solution was extracted with ethyl acetate (10 mL×3). The organic layer was washed with saturated sodium bicarbonate (10 mL×3), water (10 mL×3) and saturated sodium chloride solution (10 mL×3) sequentially, dried with anhydrous sodium sulfate, and then concentrated under reduced pressure, and purified by silica gel column chromatography to obtain Compound 4 with a yield of 47%. ¹H NMR (500 MHz, CDCl₃): δ 6.16 (d, J=3.5 Hz, 1H), 5.46 (d, J=3.0 Hz, 1H), 4.33 (t, J=10.5 Hz, 1H, H₆), 4.12-4.09 (m, 1H, H₃), 2.54-2.47 (m, 1H, H₆), 2.24-2.19 (m, 1H), 2.10-2.02 (m, 4H), 2.00 (d, J=5.5 Hz, 1H), 1.87-1.83 (m, 1H, H₅), 1.61-1.54 (m, 2H), 1.51 (s, 3H), 1.12 (s, 3H), 0.80 (d, J=4.0 Hz, 1H, H₁₆ₐ), 0.50 (d, J=4.0 Hz, 1H, H_{16b}). The ROESY spectrum shows that there are signal correlations between H₃ and H₆, H₆ and H₁₄, and H₅ and H₁₆ₐ, which confirms that the cyclopropane is in a configuration, and 3-OH and 4-OH are in a configuration. ESI-MS (m/z): [M+Na]⁺=301.2 (calcd: 301.2).

### Preparation of Compound 5

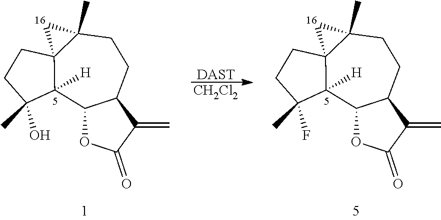

Compound 1 (124 mg, 0.50 mmol) and dichloromethane (5 mL) were added into a round-bottomed flask, protected by nitrogen, and dissolved by stirring at −78° C., into which was slowly added DAST reagent (161 mg, 1.00 mmol) dropwise, and continued to be stirred for 10-15 min after the dropwise addition was completed. The reaction solution was quenched by adding water, diluted with dichloromethane, and then washed with water (10 mL×3) and saturated sodium chloride solution (10 mL×3) sequentially, dried with anhydrous sodium sulfate, and then concentrated under reduced pressure, and purified by silica gel column chromatography to obtain Compound 5 with a yield of 55%. ¹H NMR (500 MHz, CDCl₃): δ 6.14 (d, J=3.5 Hz, 1H), 5.43 (d, J=3.0 Hz, 1H), 4.11 (t, J=10.5 Hz, 1H), 2.45-2.40 (m, 1H), 2.35-2.31 (m, 1H), 2.23-2.17 (m, 1H), 2.09-2.03 (m, 2H), 2.02-1.97 (m, 1H), 1.85-1.74 (m, 1H, H₅), 1.65 (d, J=21.5 Hz, 3H), 1.54-1.46 (m, 1H), 1.24-1.20 (m, 1H), 1.17 (s, 3H), 1.15-1.12 (m, 1H), 0.80 (d, J=4.0 Hz, 1H, H₁₆ₐ), 0.50 (d, J=4.0 Hz, 1H, H_{16b}). ¹⁹F NMR (470 MHz, CDCl₃): δ −148.17. The ROESY spectrum shows that there is a signal correlation between H₅ and H₁₆ₐ, which confirms that the cyclopropane is in a configuration. ESI-MS (m/z): [M+Na]⁺=287.2 (calcd: 287.2).

### Preparation of Compound 6

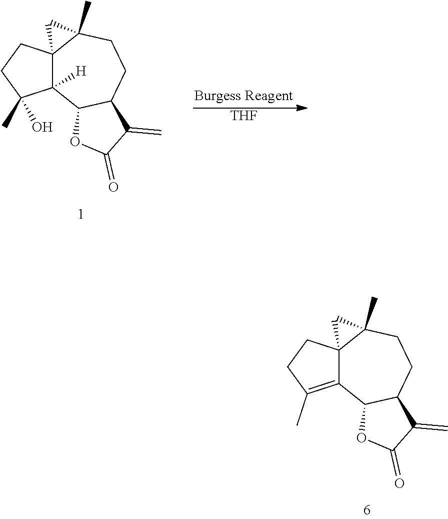

Burgess Reagent (282 mg, 1.10 mmol) and anhydrous tetrahydrofuran (10 mL) were added into a round-bottomed flask, protected by nitrogen, and placed on an ice bath. Compound 1 (262 mg, 1.00 mmol) was added. The reaction was stirred for 20 min, and then transferred to room temperature, and continued to be stirred for 3 h. The reaction solution was concentrated, and extracted with ethyl acetate (10 mL×3). The organic layer was washed with water (10 mL×3) and saturated sodium chloride solution (10 mL×3) sequentially, dried with anhydrous sodium sulfate, and then concentrated under reduced pressure, and purified by silica gel column chromatography to obtain Compound 6 with a yield of 75%. ¹H NMR (500 MHz, CDCl₃): δ 6.14 (d, J=3.5 Hz, 1H), 5.60-5.58 (m, 1H), 5.44 (d, J=3.0 Hz, 1H), 3.90 (t, J=10.0 Hz, 1H), 2.75-2.71 (m, 1H), 2.52-2.46 (m, 1H), 2.32-2.29 (m, 2H), 2.05-2.00 (m, 1H), 1.98 (s, 3H), 1.81-1.77 (m, 1H), 1.55-1.47 (m, 1H) 1.18-1.14 (m, 1H), 1.13 (s, 3H), 0.63 (d, J=5.0 Hz, 1H) , 0.51 (d, J=5.0 Hz, 1H). ESI-MS (m/z): [M+Na]⁺=267.3 (calcd: 267.3).

### Preparation of Compound 7

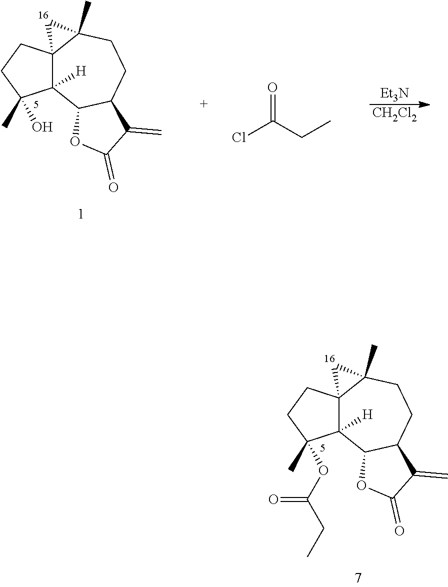

Compound 1 (262 mg, 1.00 mmol) was added into a round-bottomed flask, and protected by nitrogen, into which were added 3 mL of anhydrous dichloromethane and triethylamine (2.7 g, 27.0 mmol) sequentially, and placed on an ice bath, into which was added propionyl chloride dropwise, and warmed to room temperature for overnight reaction. The reaction solution was poured into ice water, and extracted with ethyl acetate (10 mL×3). The organic layer was washed with water (10 mL×3) and saturated sodium chloride solution (10 mL×3) sequentially, dried with anhydrous sodium sulfate, and then concentrated under reduced pressure, and purified by silica gel column chromatography to obtain Compound 7 with a yield of 51%. ¹H NMR (500 MHz, CDCl₃): δ 6.16 (d, J=3.5 Hz, 1H), 5.47 (d, J=3.0 Hz, 1H), 3.82 (t, J=10.5 Hz, 1H), 2.52-2.48 (m, 1H), 2.27-2.23 (m, 2H), 2.21-2.02 (m, 2H), 1.95-1.92 (m, 1H), 1.90-1.88 (m, 1H), 1.87-1.85 (m, 1H, H₅), 1.57 (s, 3H), 1.53-1.49 (m, 2H), 1.24 (t, J=10.0 Hz, 3H), 1.23-1.14 (m, 2H), 1.11 (s, 3H), 0.59 (d, J=4.0 Hz, 1H, H₁₆ₐ), 0.38 (d, J=4.0 Hz, 1H, H_{16b}). ROESY shows that there is a signal correlation between H₅ and H₁₆ₐ, which confirms that the cyclopropane is in a configuration. ESI-MS (m/z): [M+Na]⁺=341.2 (calcd: 341.2).

### Preparation of Compound 8

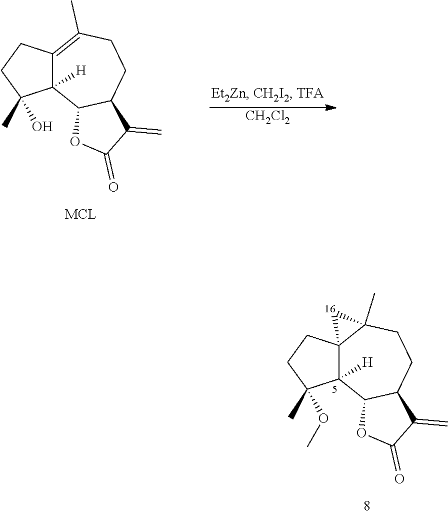

Under the condition of an ice bath and nitrogen protection, 5 mL of a diethyl zinc solution (1 M n-hexane solution) was added into 2.5 mL of dichloromethane. Trifluoroacetic acid (570 mg, 5.00 mmol) was dissolved in 0.8 mL of dichloromethane, which was added dropwise to the above solution, and the reaction was stirred for 20 min. Diiodomethane (1.34 g, 5.00 mmol) was dissolved in 0.8 mL of dichloromethane, which was added dropwise to the above solution, and the reaction was stirred for 20 min. MCL (248 mg, 1.00 mmol) was dissolved in 0.8 mL of dichloromethane, which was added to the above solution, and the reaction was stirred and reacted for 5 h. The reaction solution was quenched by adding saturated ammonium chloride, and extracted with ethyl acetate (10 mL×3). The organic layer was washed with water (10 mL×3) and saturated sodium chloride solution (10 mL×3) sequentially, dried with anhydrous sodium sulfate, and then concentrated under reduced pressure, and purified by silica gel column chromatography to obtain Compound 8 with a yield of 12%. ¹H NMR (500 MHz, CDCl₃): δ 6.14 (d, J=3.5 Hz, 1H), 5.44 (d, J=3.0 Hz, 1H), 3.83 (t, J=10.5 Hz, 1H), 3.23 (s, 3H), 2.50-2.46 (m, 1H), 2.28-2.23 (m, 1H), 2.02-1.97 (m, 4H), 1.78-1.71 (m, 1H, H₅), 1.53 (s, 3H), 1.51-1.39 (m, 2H), 1.13-1.07 (m, 1H), 1.09 (s, 3H), 0.70 (d, J=4.0 Hz, 1H, H₁₆ₐ), 0.46 (d, J=4.0 Hz, 1H, H_{16b}). The ROESY spectrum shows that there is a signal correlation between H₅ and H₁₆ₐ, which confirms that the cyclopropane is in a configuration. ESI-MS (m/z): [M+Na]⁺=299.2 (calcd: 299.2).

### Example 3: Stability Test of Compounds 1

2.00 mg of Compounds 1-8 were precisely weighed. The samples were dissolved in 500 μL of chromatographic methanol, into which were added 1500 μL of artificial gastric juice, mixed well and then dissolved completely by ultrasound. 50 μL of the above solutions were aspirated precisely, diluted by adding 200 μL of chromatographic methanol, filtered and analyzed by HPLC, wherein the HPLC analysis conditions were mobile phase: 65% methanol -35% water, flow rate: 0.8 mL/min, and column temperature: 25° C., and the initial peak areas were recorded. The above solutions were placed on a constant temperature water bath at 37° C., sampled at 8 h, 16 h and 24 h respectively, and analyzed by HPLC. By calculating the peak areas respectively, the stability data of the corresponding samples in artificial gastric juice environment at 8 h, 16 h and 24 h can be obtained.

As shown in Table 1, the stabilities of Compound 1˜8 are significantly improved compared with that of arglabin and dehydrocostus lactone.

TABLE 1
Stability of compound in artificial gastric juice environment

| | Non-degradation | Non-degradation | Non-degradation |
|---|---|---|---|
| | percentage at | percentage at | percentage at |
| Group | 8 h | 16 h | 24 h |
| Compound 1 | 99.08% | 98.76% | 98.16% |
| Compound 2 | 90.92% | 86.62% | 78.26% |
| Compound 3 | 99.98% | 99.87% | 97.77% |
| Compound 4 | 98.15% | 96.84% | 96.21% |
| Compound 5 | 99.91% | 99.85% | 99.84% |
| Compound 6 | 99.89% | 96.80% | 95.72% |
| Compound 7 | 99.31% | 97.85% | 97.65% |
| Compound 8 | 98.36% | 95.58% | 94.08% |
| arglabin | 50.14% | 48.70% | 43.40% |
| dehydrocostus | 68.91% | 65.42% | 57.86% |
| lactone | | | |

### Example 4: Preparation of Prodrug (Prodrugs Include Salts)

### Preparation of Compound 9

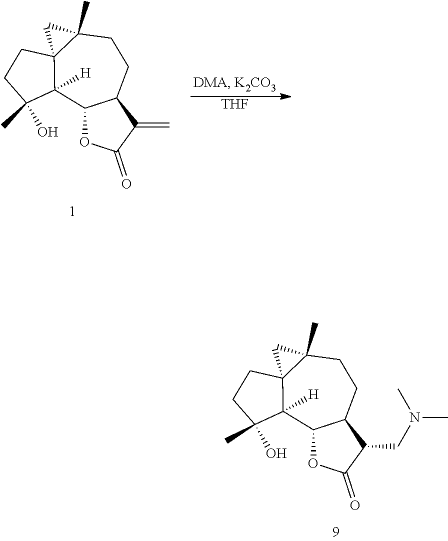

Compound 1 (262 mg, 1.00 mmol), dichloromethane (30 mL), dimethylamine hydrochloride (815 mg, 10.00 mmol) and potassium carbonate (2764 mg, 20 mmol) were added into a round-bottomed flask, and stirred for 4 h. The reaction solution was filtered, washed with water (10 mL×3) and saturated sodium chloride solution (10 mL×3) sequentially, dried with anhydrous sodium sulfate, and then concentrated under reduced pressure, and purified by silica gel column chromatography (petroleum ether: ethyl acetate: triethylamine =1: 1: 0.02) to obtain Compound 9 with a yield of 85%. ¹H NMR (500 MHz, CDCl₃): δ 3.92 (t, J=10.5 Hz, 1H), 3.55-3.47 (m, 1H), 3.33-3.29 (m, 1H), 2.38-2.34 (m, 1H), 2.28 (s, 6H), 2.21-2.16 (m, 2H), 1.98 (d, J=15.0 Hz, 2H), 1.88 (t, J=5.0 Hz, 2H), 1.72 (d, J=10.0 Hz, 1H), 1.60-1.52 (m, 2H), 1.50 (s, 3H), 1.20 (t, J=5.0 Hz, 1H), 1.12 (s, 3H), 1.08-1.04 (m, 1H), 0.76 (d, J=5.0 Hz, 1H), 0.53 (d, J=5.0 Hz, 1H). ESI-MS (m/z): [M+Na]⁺=330.2 (calcd: 330.2).

### Preparation of Compound 10

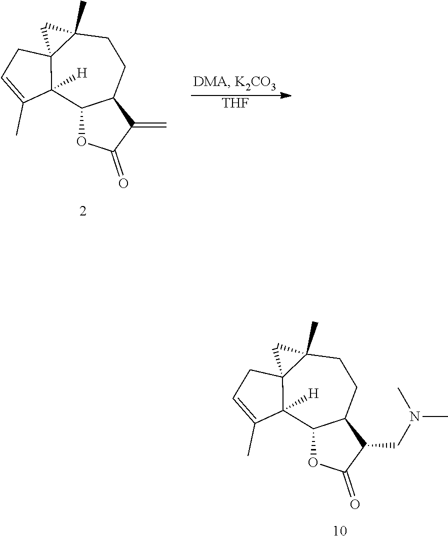

The preparation method is the same as that of Compound 9 with Compound 2 and dimethylamine hydrochloride as starting materials, and the yield of Compound 10 is 80%. ¹H NMR (500 MHz, CDCl₃): δ 5.52-5.46 (m, 1H), 3.98 (t, J=10.0 Hz, 1H),3.41-3.32 (m, 1H), 3.20-3.16 (m, 1H), 2.98-2.92 (m, 1H),2.28 (s, 6H), 2.22-2.19 (m, 1H), 2.10 (d, J=10.0 Hz, 1H), 2.06-2.02 (m, 1H), 1.98-1.94 (m, 1H), 1.91 (s, 3H), 1.84-1.80 (m, 2H), 1.62-1.56 (m, 1H), 1.19 (s, 3H), 1.14-1.08 (m, 1H), 0.69 (d, J=5.0 Hz, 1H), 0.51 (d, J=5.0 Hz, 1H). ESI-MS (m/z): [M+Na]⁺=312.2 (calcd: 312.2).

### Preparation of Compound 11

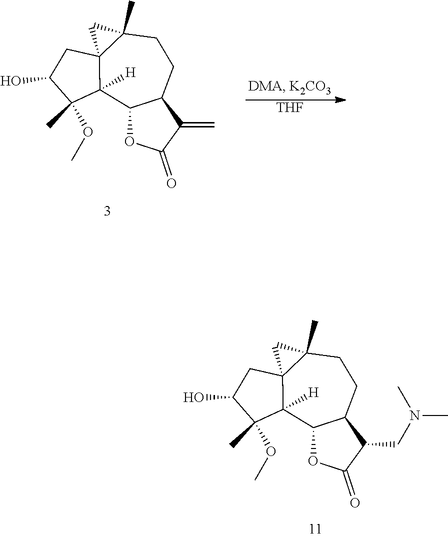

The preparation method is the same as that of Compound 9 with Compound 3 and dimethylamine hydrochloride as starting materials, and the yield of Compound 11 is 95%. ¹H NMR (500 MHz, CDCl₃): δ 3.96 (t, J=10.0 Hz, 1H), 3.28-3.23 (m, 1H), 3.15-3.10 (m, 1H), 2.94 (s, 3H), 2.70-2.65 (m, 1H), 2.28 (s, 6H), 2.03-1.99 (m, 1H), 1.91-1.88 (m, 1H), 1.86-1.76 (m, 4H), 1.62-1.56 (m, 1H), 1.48 (s, 3H), 1.28-1.24 (m, 1H), 1.12 (s, 3H), 1.09-1.05 (m, 1H), 0.70 (d, J=5.0 Hz, 1H), 0.54 (d, J=5.0 Hz, 1H). ESI-MS (m/z): [M+Na]⁺=360.4 (calcd: 360.5).

### Preparation of Compound 12

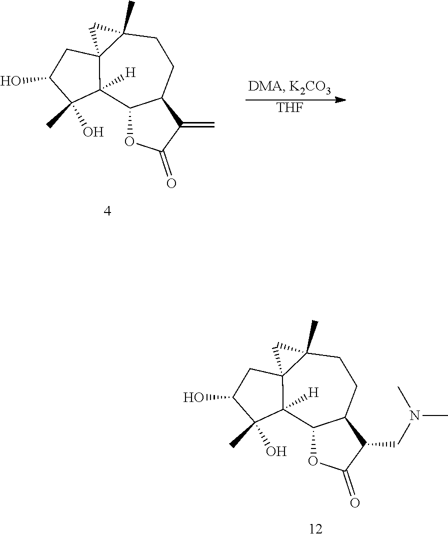

The preparation method is the same as that of Compound 9 with Compound 4 and dimethylamine hydrochloride as starting materials, and the yield of Compound 12 is 90%. ¹H NMR (500 MHz, CDCl₃): δ 3.94 (t, J=10.0 Hz, 1H), 3.38-3.36 (m, 1H), 3.23-3.18 (m, 1H), 3.19-3.15 (m, 2H), 2.78-2.70 (m, 1H), 2.28 (s, 6H), 2.13-2.09 (m, 1H), 1.97-1.92 (m, 1H), 1.90-1.86 (m, 1H), 1.82-1.79 (m, 1H), 1.77-1.75 (m, 1H), 1.62-1.59 (m, 1H), 1.51 (s, 3H), 1.18 (s, 3H), 1.11-1.07 (m, 1H), 0.70 (d, J=5.0 Hz, 1H), 0.48 (d, J=5.0 Hz, 1H). ESI-MS (m/z): [M+Na]⁺=346.2 (calcd: 346.2).

### Preparation of Compound 13

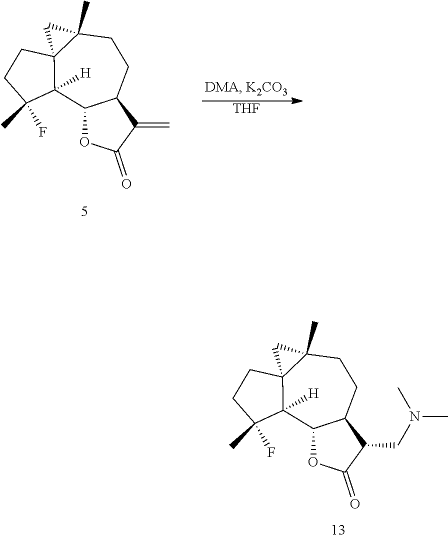

The preparation method is the same as that of Compound 9 with Compound 5 and dimethylamine hydrochloride as starting materials, and the yield of Compound 13 is 83%. ¹H NMR (500 MHz, CDCl₃): δ 3.96 (t, J=10.0 Hz, 1H), 3.35-3.30 (m, 2H), 2.80-2.76 (m, 1H), 2.30 (s, 6H), 2.29-2.19 (m, 2H), 2.08 (t, J=10.0 Hz, 1H), 1.96-1.90 (m, 1H), 1.84-1.82 (m, 2H), 1.81-1.77 (m, 2H), 1.58 (d, J=21.5 Hz, 3H) 1.46-1.39 (m, 1H), 1.18 (s, 3H), 1.16-1.12 (m, 1H), 0.62 (d, J=5.0 Hz, 1H), 0.58 (d, J=5.0 Hz, 1H). ESI-MS (m/z): [M+Na]⁺=332.4 (calcd: 332.4).

### Preparation of Compound 14

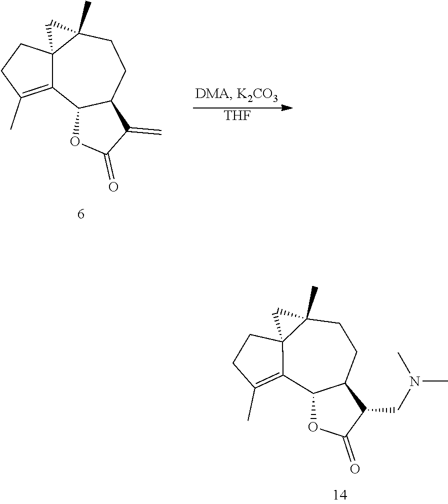 [SMILES: C=C1C(=O)O[C@@H]2C3=C(C)CC[C@]34C[C@]4(C)CC[C@@H]12 ; CC1=C2[C@H]3OC(=O)[C@@H](CN(C)C)[C@@H]3CC[C@@]3(C)C[C@@]23CC1 ; "THF"]

The preparation method is the same as that of Compound 9 with Compound 6 and dimethylamine hydrochloride as starting materials, and the yield of Compound 14 is 91%. ¹H NMR (500 MHz, CDCl3): δ 3.99 (t, J=10.0 Hz, 1H),3.41-3.36 (m, 1H), 3.20-3.18 (m, 1H), 2.82-2.77 (m, 1H), 2.28 (s, 6H), 2.22-2.19 (m, 1H), 2.16-2.10 (m, 2H), 1.98-1.94 (m, 1H), 1.91 (s, 3H), 1.84-1.80 (m, 1H), 1.78-1.75 (m, 1H), 1.62-1.56 (m, 2H), 1.15 (s, 3H), 1.11-1.07 (m, 1H), 0.64 (d, J=5.0 Hz, 1H), 0.50 (d, J=5.0 Hz, 1H). ESI-MS (m/z): [M+Na]⁺=312.4 (calcd: 312.4).

### Preparation of Compound 15

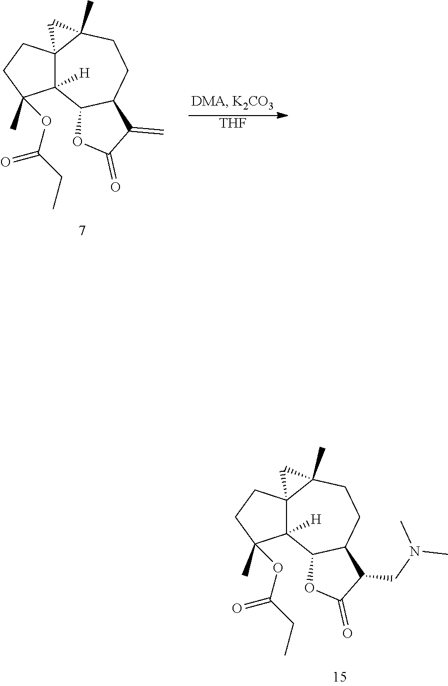

The preparation method is the same as that of Compound 9 with Compound 7 and dimethylamine hydrochloride as starting materials, and the yield of Compound 15 is 80%. ¹H NMR (500 MHz, CDCl3): δ 3.92 (t, J=10.0 Hz, 1H), 3.40-3.30 (m, 2H), 2.83-2.79 (m, 1H), 2.30 (s, 6H),2.19-2.13 (m, 2H), 2.04-1.97 (m, 2H), 1.95-1.90 (m, 2H), 1.86-1.82 (m, 1H), 1.76-1.73 (m, 1H), 1.60 (s, 3H), 1.56-1.52 (m, 1H), 1.33-1.25 (m, 1H), 1.14 (s, 3H), 1.02 (t, J=10.0 Hz, 3H), 1.00-0.95 (m, 1H), 0.88-0.82 (m, 1H), 0.48 (d, J=5.0 Hz, 1H), 0.40 (d, J=5.0 Hz, 1H). ESI-MS (m/z): [M+Na]⁺=386.5 (calcd: 386.5).

### Preparation of Compound 16

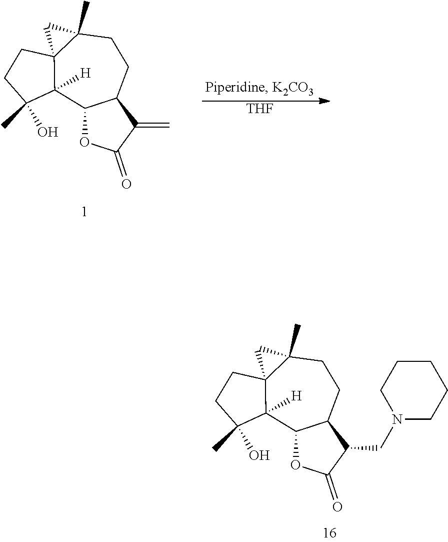

The preparation method is the same as that of Compound 9 with Compound 1 and piperidine as starting materials, and the yield of Compound 16 is 89%. ¹H NMR (500 MHz, CDCl₃): δ 3.79 (t, J=10.5 Hz, 1H), 2.79-2.75 (m, 1H), 2.56-2.53 (m, 1H), 2.46-2.40 (m, 3H), 2.39-2.32 (m, 3H), 2.19-2.08 (m, 4H), 1.94-1.85 (m, 3H), 1.77 (d, J=10.5 Hz, 1H), 1.54 (s, 6H), 1.45-1.39 (m, 4H), 1.12 (s, 3H), 1.09-1.04 (m, 1H), 0.79 (d, J=4.0 Hz, 1H), 0.52 (d, J=4.0 Hz, 1H). ESI-MS (m/z): [M+Na]⁺=370.2 (calcd: 370.2).

### Preparation of Compound 17

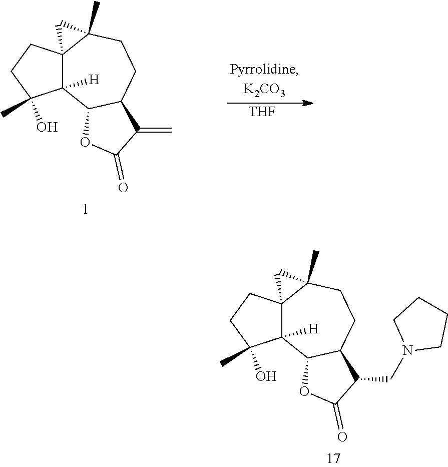

The preparation method is the same as that of Compound 9 with Compound 1 and tetrahydropyrrole as starting materials, and the yield of Compound 17 is 87%. ¹H NMR (500 MHz, CDCl₃): δ 3.79 (t, J=10.5 Hz, 1H), 2.89-2.80 (m, 2H), 2.57-2.51 (m, 4H), 2.37-2.32 (m, 1H), 2.15-2.06 (m, 3H), 1.94-1.87 (m, 3H), 1.78-1.76 (m, 5H), 1.55 (s, 3H), 1.51-1.39 (m, 3H), 1.11 (s, 3H), 1.09-1.03 (m, 1H), 0.77 (d, J=4.0 Hz, 1H), 0.51 (d, J=4.0 Hz, 1H). ESI-MS (m/z): [M+Na]⁺=356.3 (calcd: 356.2).

### Preparation of Compound 18

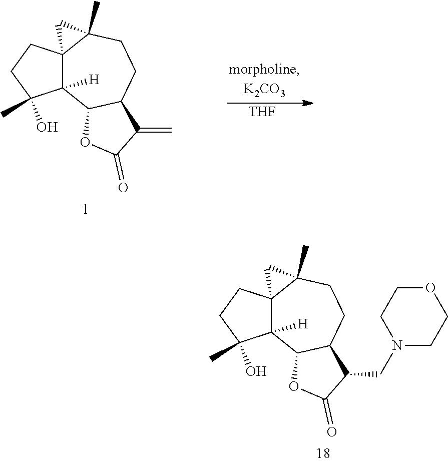

The preparation method is the same as that of Compound 9 with Compound 1 and tetrahydropyrrole as starting materials, and the yield of Compound 18 is 76%. ¹H NMR (500 MHz, CDCl₃): δ 3.81 (t, J=10.5 Hz, 1H), 3.74-3.67 (m, 3H), 2.82-2.79 (m, 1H), 2.65-2.61 (m, 1H), 2.53-2.44 (m, 5H), 2.40-2.35 (m, 1H), 2.21-2.17 (m, 1H), 2.13-2.07 (m, 2H), 1.95-1.86 (m, 3H), 1.78 (d, J=10.5 Hz, 1H), 1.55 (s, 3H), 1.52-1.39 (m, 2H), 1.31-1.27 (m, 1H), 1.12 (s, 3H), 1.09-1.04 (m, 1H), 0.80 (d, J=4.0 Hz, 1H), 0.54 (d, J=4.0 Hz, 1H). ESI-MS (m/z): [M+Na]⁺=372.2 (calcd: 372.2).

### Preparation of Compound 19

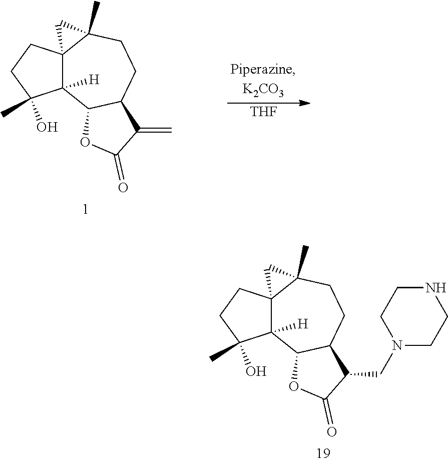

The preparation method is the same as that of Compound 9 with Compound 1 and piperazine as starting materials, and the yield of Compound 19 is 74%. ¹H NMR (500 MHz, CDCl₃): δ 3.79 (t, J=10.5 Hz, 1H), 2.82-2.79 (m, 1H), 2.64-2.60 (m, 1H), 2.55-2.42 (m, 5H), 2.38-2.34 (m, 2H), 2.29 (s, 3H), 2.20-2.16 (m, 1H), 2.15-2.05 (m, 2H), 1.93-1.83 (m, 3H), 1.76 (d, J=10.5 Hz, 1H), 1.54 (s, 3H), 1.49-1.38 (m, 2H), 1.29-1.25 (m, 1H), 1.12 (s, 3H), 1.09-1.03 (m, 1H), 0.78 (d, J=4.0 Hz, 1H), 0.52 (d, J=4.0 Hz, 1H). ESI-MS (m/z): [M+Na]⁺=371.2 (calcd: 371.2).

### Preparation of Compound 20

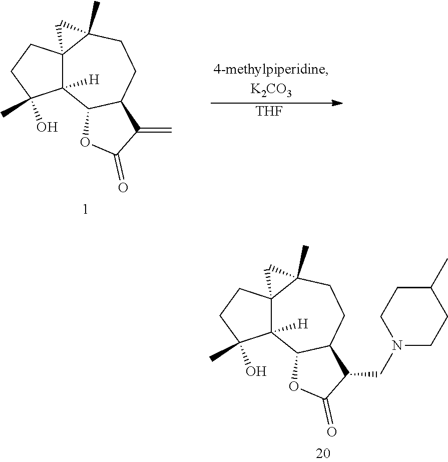

The preparation method is the same as that of Compound 9 with Compound 1 and piperazine as starting materials, and the yield of Compound 20 is 74%. ¹H NMR (500 MHz, CDCl₃): δ 3.79 (t, J=10.5 Hz, 1H), 2.83-2.74 (m, 3H), 2.59-2.55 (m, 1H), 2.39-2.35 (m, 2H), 2.20-2.07 (m, 4H), 1.96-1.83 (m, 4H), 1.78 (d, J=10.5 Hz, 1H), 1.55 (s, 3H), 1.51-1.23 (m, 5H), 1.21-1.15 (m, 2H), 1.13 (s, 3H), 1.10-1.05 (m, 1H), 0.93 (d, J=6.5 Hz, 3H), 0.79 (d, J=4.0 Hz, 1H), 0.52 (d, J=4.0 Hz, 1H). ESI-MS (m/z): [M+Na]⁺=384.3 (calcd: 384.3).

### Preparation of Compound 21

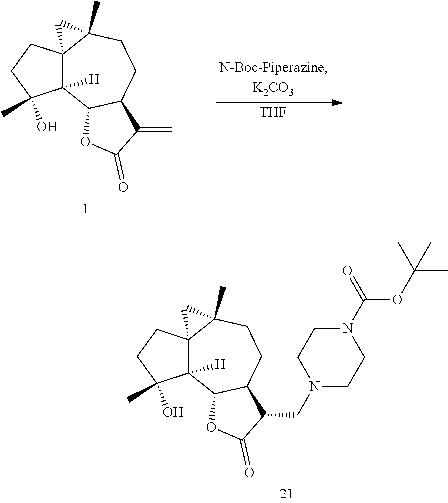

The preparation method is the same as that of Compound 9 with Compound 1 and N-Boc-piperazine as starting materials, and the yield of Compound 21 is 65%. ¹H NMR (500 MHz, CDCl₃): δ 3.81 (t, J=10.5 Hz, 1H), 2.83-2.79 (m, 1H), 2.66-2.62 (m, 2H), 2.42-2.35 (m, 4H), 2.21-2.16 (m, 1H), 2.13-2.07 (m, 2H), 1.94-1.84 (m, 3H), 1.78 (d, J=10.5 Hz, 1H), 1.55 (s, 3H), 1.48 (s, 9H), 1.46-1.44 (m, 1H), 1.40-1.39 (m, 1H), 1.32-1.25 (m, 3H), 1.13 (s, 3H), 1.10-1.04 (m, 2H), 0.92-0.85 (m, 1H), 0.80 (d, J=4.0 Hz, 1H), 0.54 (d, J=4.0 Hz, 1H). ESI-MS (m/z): [M+Na]⁺=471.3 (calcd: 471.3).

### Preparation of Hydrochloride 22 of Compound 9

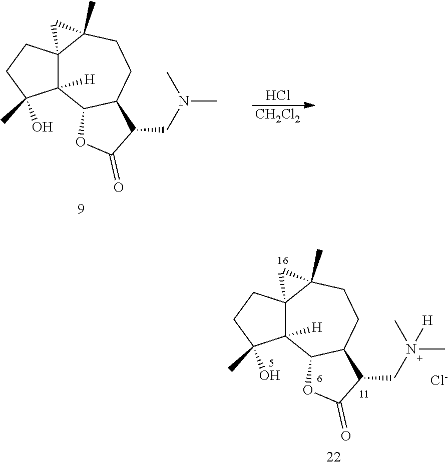

Compound 9 (307 mg, 1 mmol) was dissolved in dichloromethane (2 mL), and stirred at room temperature for 2 h, into which was then added a hydrochloric acid solution dropwise until the pH value was 4-5, and filtered. The resulting solid was washed with dichloromethane to obtain a white solid, i.e., the hydrochloride (Compound 22) of Compound 9, with a yield of 90%. ¹H NMR (500 MHz, CD₃OD): δ 4.15-4.10 (m, 1H, H₆), 3.42-3.37 (m, 1H),3.31-3.26 (m, 1H), 3.04-2.98 (m, 1H, H₁₁),2.91 (s, 6H), 2.21-2.12 (m, 2H), 2.02 (d, J=15.0 Hz, 2H), 1.88 (t, J=5.0 Hz, 2H), 1.78 (d, J=10.0 Hz, 1H, H₅), 1.64-1.54 (m, 2H), 1.52 (s, 3H), 1.26 (t, J=5.0 Hz, 1H), 1.14 (s, 3H), 1.10-1.05 (m, 1H), 0.76 (d, J=5.0 Hz, 1H, H₁₆ₐ), 0.53 (d, J=5.0 Hz, 1H, H_{16b}). The ROESY spectrum shows that there are signal correlations between the hydrogens of H₅ and H₁₆ₐ, as well as the hydrogens of H₆ and H₁, which confirms that the cyclopropane is in α configuration, and 11-His in β configuration. ESI-MS (m/z): [M+H]⁺=308.2 (calcd: 308.2).

### Preparation of Fumarate 23 of Compound 9

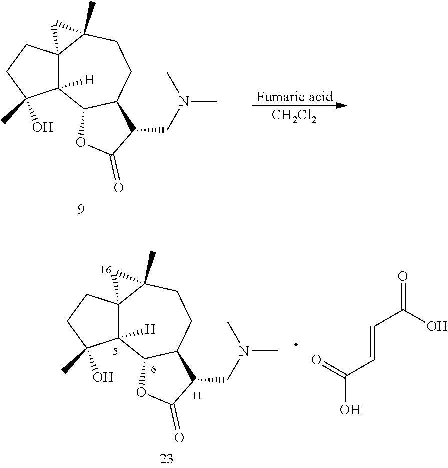

The fumarate Compound 23 was prepared by using fumaric acid instead of hydrochloric acid according to the preparation method of the hydrochloride of Compound 9. The yield was 80%. ¹H NMR (500 MHz, CD₃OD): δ 6.72 (s, 2H), 4.13 (t, J=10.0 Hz, 1H, H₆), 3.41-3.39 (m, 1H), 3.30-3.27 (m, 1H), 3.03-2.98 (m, 1H, Hn), 2.91 (s, 6H), 2.21-2.12 (m, 2H), 1.94-1.91 (m, 1H), 1.88 (t, J=5.0 Hz, 2H), 1.84-1.80 (m, 1H), 1.78 (d, J=15.0 Hz, 1H, H₅), 1.64-1.54 (m, 2H), 1.52 (s, 3H), 1.14 (s, 3H), 1.10-1.04 (m, 1H), 0.75 (d, J=5.0 Hz, 1H, H₁₆ₐ), 0.52 (d, J=5.0 Hz, 1H, H_{16b}). The ROESY spectrum shows that there are signal correlations between H₅ and H₁₆ₐ, as well as the H₆ and H₁₁, which confirms that the cyclopropane is in a configuration, and 11-H is in β configuration. ESI-MS (m/z): [M+H]⁺=308.2 (calcd: 308.2).

### Preparation of Hydrochloride 24 of Compound 10

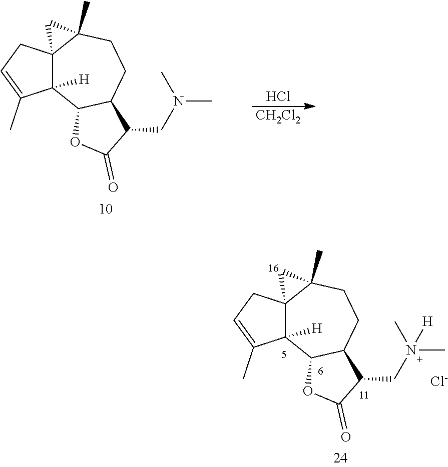

Using Compound 10 as the starting material, Compound 24 could be prepared according to the preparation method of the hydrochloride of Compound 9, and the yield was 95%. ¹H NMR (500 MHz, CD₃OD): δ 5.62-5.58 (m, 1H), 4.19 (t, J=10.0 Hz, 1H, H₆), 3.51-3.46 (m, 1H), 3.40-3.36 (m, 1H), 3.12-3.07 (m, 1H, H₁₁), 2.98 (s, 6H), 2.82-2.79 (m, 1H), 2.30 (d, J=10.0 Hz, 1H), 2.26-2.12 (m, 1H), 1.98-1.94 (m, 1H), 1.91 (s, 3H), 1.84-1.82 (m, 2H), 1.80-1.78 (m, 1H, H₅), 1.66-1.59 (m, 1H), 1.17 (s, 3H), 1.16-1.10 (m, 1H), 0.61 (d, J=5.0 Hz, 1H, H₁₆ₐ), 0.48 (d, J=5.0 Hz, 1H, H_{16b}). The ROESY spectrum shows that there are signal correlations between H₅ and H₁₆ₐ, as well as the H₆ and H₁₁, which confirms that the cyclopropane is in a configuration, and 11-His in β configuration. ESI-MS (m/z): [M+H]⁺=290.2 (calcd: 290.2).

### Preparation of Hydrochloride 25 of Compound 11

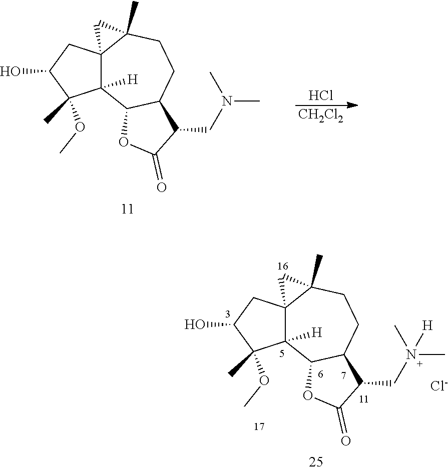

Using Compound 11 as the starting material, Compound 25 could be prepared according to the preparation method of the hydrochloride of Compound 9, and the yield was 95%. ¹H NMR (500 MHz, CD₃OD): δ 4.39 (t, J=10.0 Hz, 1H, H₆), 3.48-3.43 (m, 1H, H₇), 3.40-3.36 (m, 1H), 3.34 (s, 3H, H₁₇), 3.10-3.05 (m, 1H, H₁₁), 2.98 (s, 6H), 2.23-2.19 (m, 1H, H₃), 2.11-2.08 (m, 1H), 1.87-1.80 (m, 3H), 1.79-1.77 (m, 1H, H₅), 1.65-1.56 (m, 1H), 1.51 (s, 3H), 1.27-1.25 (m, 1H), 1.15 (s, 3H), 1.13-1.08 (m, 1H), 0.74 (d, J=5.0 Hz, 1H, H₁₆ₐ), 0.51 (d, J=5.0 Hz, 1H, H_{16b}). ESI-MS (m/z): [M+H]⁺=338.2 (calcd: 338.2).

### Preparation of Hydrochloride 26 of Compound 12

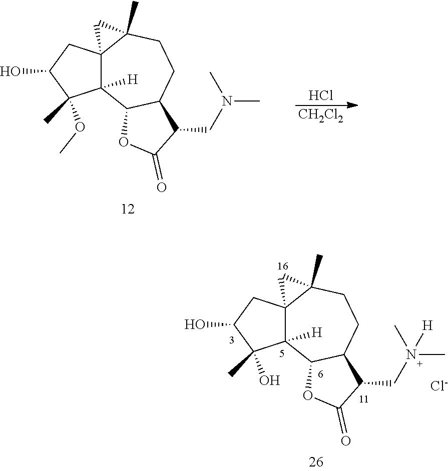

Using Compound 12 as the starting material, Compound 26 could be prepared according to the preparation method of the hydrochloride of Compound 9, and the yield was 85%. ¹H NMR (500 MHz, CD₃OD): δ 4.47 (t, J=10.0 Hz, 1H, H₆), 3.88-3.86 (m, 1H), 3.63-3.58 (m, 1H), 3.49-3.45 (m, 2H), 3.10-3.06 (m, 1H, H₁₁), 3.00 (s, 6H), 2.23-2.19 (m, 1H), 1.97-1.92 (m, 1H), 1.90-1.81 (m, 3H), 1.66-1.59 (m, 1H, Hs), 1.52 (s, 3H), 1.14 (s, 3H), 1.12-1.10 (m, 1H), 0.73 (d, J=5.0 Hz, 1H), 0.50 (d, J=5.0 Hz, 1H). ESI-MS (m/z): [M+H]⁺=324.2 (calcd: 324.2).

### Preparation of Hydrochloride 27 of Compound 13

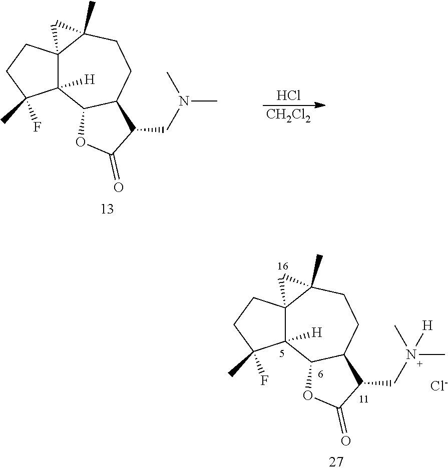

Using Compound 13 as the starting material, Compound 27 could be prepared according to the preparation method of the hydrochloride of Compound 9, and the yield was 83%. ¹H NMR (500 MHz, CD₃OD): δ 4.36 (t, J=10.0 Hz, 1H, H₆), 3.50-3.40 (m, 2H), 3.15-3.11 (m, 1H, H₁₁), 3.00 (s, 6H), 2.29-2.19 (m, 2H), 2.11 (t, J=10.0 Hz, 1H), 2.06-2.00 (m, 1H), 1.94-1.87 (m, 2H), 1.84-1.82 (m, 1H), 1.81-1.78(m, 1H, H₅) 1.60 (d,J=21.5 Hz, 3H) 1.60-1.51 (m, 1H), 1.20 (s, 3H), 1.18-1.15 (m, 1H), 0.54 (d, J=5.0 Hz, 1H, H₁₆ₐ), 0.50 (d, J=5.0 Hz, 1H, H_{16b}). ESI-MS (m/z): [M+H]⁺=310.2 (calcd: 310.2).

### Preparation of Hydrochloride 28 of Compound 14

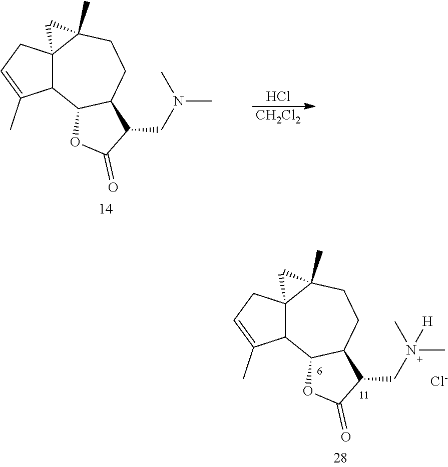

Using Compound 14 as the starting material, Compound 28 could be prepared according to the preparation method of the hydrochloride of Compound 9, and the yield was 80%. ¹H NMR (500 MHz, CD₃OD): δ 4.19 (t, J=10.0 Hz, 1H, H₆), 3.51-3.46 (m, 1H), 3.40-3.36 (m, 1H), 3.12-3.07 (m, 1H, H₁₁), 2.98 (s, 6H), 2.82-2.79 (m, 1H), 2.26-2.12 (m, 2H), 1.98-1.94 (m, 1H), 1.91 (s, 3H), 1.84-1.80 (m, 2H), 1.66-1.59 (m, 2H), 1.17 (s, 3H), 1.16-1.10 (m, 1H), 0.61 (d, J=5.0 Hz, 1H), 0.48 (d, J=5.0 Hz, 1H). ESI-MS (m/z): [M+H]⁺=290.2 (calcd: 290.2).

### Preparation of Hydrochloride 29 of Compound 15

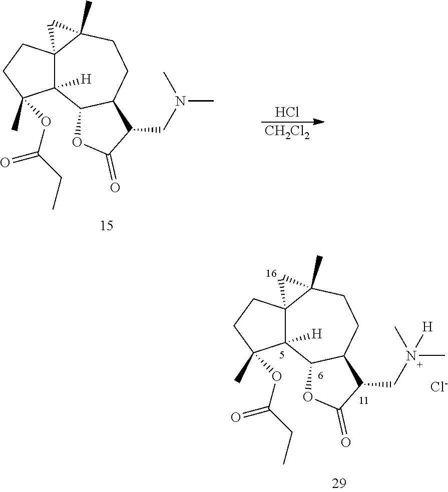

Using Compound 15 as the starting material, Compound 29 could be prepared according to the preparation method of the hydrochloride of Compound 9, and the yield was 76%. ¹H NMR (500 MHz, CD₃OD): δ 4.22 (t, J=10.0 Hz, 1H, H₆), 3.50-3.38 (m, 2H), 3.13-3.09 (m, 1H, H₁₁), 2.99 (s, 6H), 2.69-2.63 (m, 2H), 2.24-2.17 (m, 2H), 1.99-1.93 (m, 2H), 1.86-1.82 (m, 1H, H₅), 1.68-1.59 (m, 1H), 1.54-1.45 (m, 1H), 1.36-1.30 (m, 1H), 1.27 (s, 3H), 1.14 (s, 3H), 1.06 (t, J=10.0 Hz, 3H), 1.04-1.00 (m, 1H), 0.93-0.88 (m, 1H), 0.42 (d, J=5.0 Hz, 1H, H₁₆ₐ), 0.36 (d, J=5.0 Hz, 1H, H_{16b}). ESI-MS (m/z): [M+H]⁺=364.2 (calcd: 364.2).

### Example 5: Compound 23 is Converted to Compound 1 in Plasma and HEPES

Experimental method

Formulation of HEPES 7.4 solution: 1.6 g of NaCl, 0.074 g of KCl, 0.027 g of Na₂HPO₄, 0.2 g of glucose and 1 g of a 4-hydroxyethyl piperazine ethanesulfonic acid (HEPES) solution were placed in 90 mL of distilled water, the pH of which was adjusted to 7.4 with 0.5 M NaOH, and then the volume was adjusted to 100 mL with distilled water.

Preparation of plasma: Mouse plasma was placed in an EP tube filled with heparin sodium in advance, centrifuged at 8000 rpm at 4° C. for 10 min, and the supernatant was taken.

Sample analysis: 0.6 mg of Compound 23 was dissolved in 250 ul deionized water. 250 μL of the mouse serum or the HEPES7.4 solution was added into the sample, incubated at 37° C., and sampled at different time points, respectively. 20 μL of samples were taken into EP tubes, into which was added 60 μL of methanol, vortexed to mix well, and centrifuged at 12000 rpm at 4° C. for 10 min. The supernatants were taken at 1 h, 2 h, 4 h, 8 h and 12 h, respectively. The samples were analyzed by HPLC with an injection volume of 10 μL, and the corresponding peak areas were recorded. The chromatographic conditions were as follows: the chromatographic column was Hanbang C18 (4.6×250 mm, 5 um); the mobile phase was acetonitrile: 10 mmol/mL of ammonium formate solution =60: 40; the flow rate was 1.0 mL/min; the detection wavelength was 210 nm; and the column temperature was 30° C.

### Experimental Result

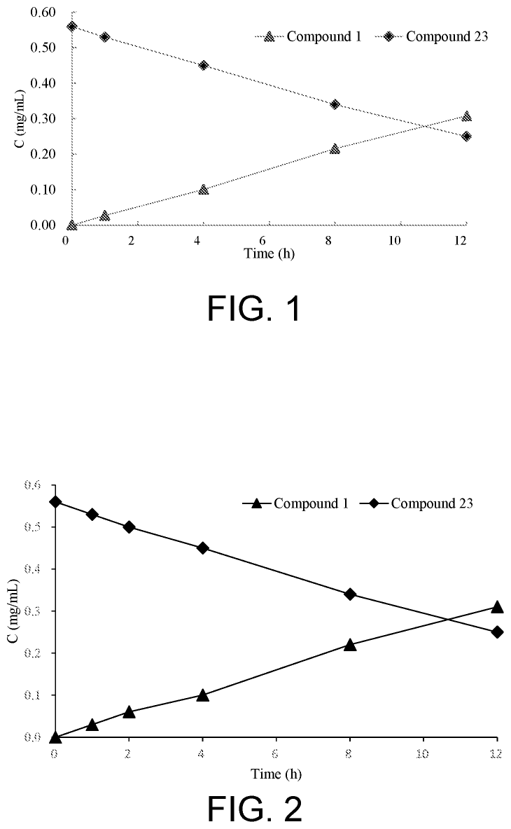

As shown in FIGS. 1, at 1 h, 2 h, 4 h, 8 h and 12 h, the contents of Compound 1 in the HEPES buffer solution are gradually increased, and the contents are 5.36%, 11.05%, 19.64%, 39.29% and 55.36%, respectively. The experimental result shows that, Compound 23 can be converted to the prototype Compound 1 as a prodrug in the HEPES buffer solution. As shown in FIGS. 2, at 1 h, 2 h, 4 h, 8 h and 12 h, the contents of Compound 1 in the mouse plasma are gradually increased, and the contents are 5.77%, 11.05%, 18.86%, 39.42% and 55.88%, respectively. The experimental result shows that, Compound 23 can be converted to the prototype Compound 1 as a prodrug in the mouse plasma.

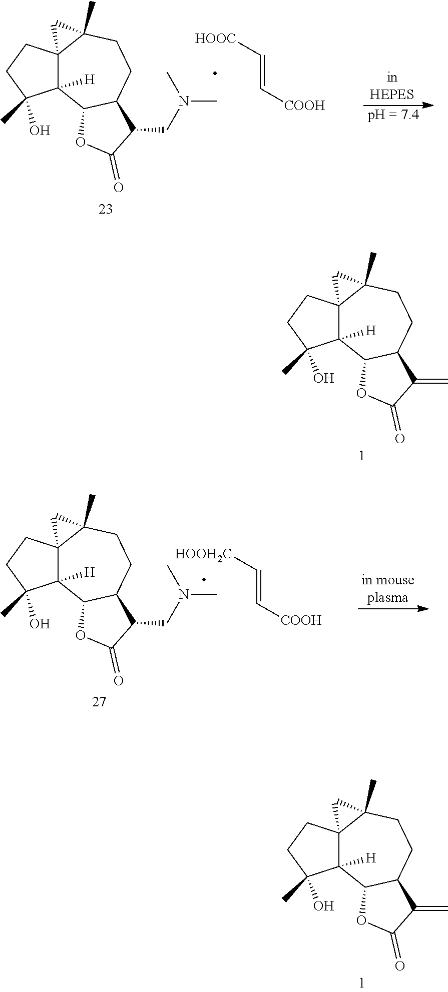

Similarly, other prodrug compounds can be converted into corresponding parent drug compounds in plasma and HEPES.

### Example 6: Solubility test of prototype and its prodrug in water

20 μg of Compounds 1-8 and 10 mg of Compounds 22-29 were precisely weighed, respectively, added into 1 mL of deionized water, and completely dissolved by ultrasound. Saturated solutions were formulated, filtered and analyzed by HPLC with a sample volume of 1 μL, 3 μL, 5 μL, 10 μL, 15 UL and 20 μL sequentially, and the standard curves of the corresponding compounds were drawn.

Unsaturated solutions of the above compounds were formulated, dissolved by ultrasound for 4 h, and placed on a water bath at 37° C. to stand for 1 h. The resulting unsaturated solutions were centrifuged. 30 μL of supernatants were removed, diluted by adding 200 μL of deionized water, and then filtered. The samples were analyzed by HPLC, and the solubilities of test Compounds 1-8 and 22-29 were obtained by substituting the relevant data into the above measured standard curves.

TABLE 2
Water solubility of compound

| | Water | Salt of 13,13-N,N- | Water |
|---|---|---|---|
| | solubility | dimethyl derivative | solubility |
| Derivative | μg/mL | prodrug | mg/mL |
| Compound 1 | 1140.00 | Compound 22 | 320.70 |
| | | Compound 23 | 310.40 |
| Compound 2 | 0.80 | Compound 24 | 96.60 |
| Compound 3 | 35.48 | Compound 25 | 220.80 |
| Compound 4 | 100.82 | Compound 26 | 248.50 |
| Compound 5 | 25.88 | Compound 27 | 208.40 |
| Compound 6 | 2.58 | Compound 28 | 120.50 |
| Compound 7 | 17.66 | Compound 29 | 194.60 |
| Compound 8 | 8.52 | | |
| arglabin | 7.94 | dehydrocostus lactone | 44.82 |

As shown in Table 1, the water solubilities of the prodrug salts 22˜29 are at least more than 100 times higher than that of arglabin, dehydrocostunolide and the corresponding parent drugs.

### Example 7: Comparison of Pharmacokinetic Property of Compounds 1 and 23 at Equimolar Dose

### Experimental Material

### Experimental Reagent

The drugs of the present application were prepared according to the above examples; Tolbutamide (an internal standard, IS), Dalian Meilun Biotechnology Co., Ltd.; Dimethyl sulfoxide, Shanghai Titan Technology Co., Ltd.; Normal saline, Chenxin Pharmaceutical Co., Ltd.; Sodium carboxymethyl cellulose, Aladdin Company; Methanol, Acetonitrile and Formic acid, Merck Company; and Pure water, Hangzhou Wahaha Group Co., Ltd.

### Experimental Apparatus

Freezing centrifuge, Eppendorf Company; Vortex oscillator, Scientific Industries Company; Numerical control ultrasonic cleaner, Kunshan Ultrasonic Instrument Co., Ltd.; Electronic balance, Sartorius Company; Magnetic stirrer, IKA Company; Electronic balance, Changzhou Lucky Electronic Equipment Co., Ltd.; and H-Class/Xevo TQ-S micro LC-MS, Waters Company.

### Experimental Animal

SPF male SD rats, weighing 200+20 g, were provided by Qinglong Mountain Animal Farm, Jiangning District, Nanjing. After purchase, the animals were kept under the condition of ambient temperature of 23˜26° C. and humidity of 40˜60% for 7 days, during which they were free to eat and drink. The animal production license number was SCXK (Zhe) 2019-0002.

### Experimental Method

### Establishment of UPLC-MS/MS Determination Method

Chromatographic conditions: Waters Acquity UPLC® BEH C¹⁸ column (2.1×50 mm, 1.7 μm) was used; and gradient elution was performed with 0.1% formic acid aqueous solution as mobile phase A and acetonitrile as mobile phase B (0-1.0 min, 5%-30% B; 1.0-2.0 min, 30%-80% B; 2.0-3.0 min, 80%-80% B; 3.0-4.0 min, 80%-5%B; 4.0-5.0 min, 5%-5% B), total running time: 5 min; flow rate: 0.3 mL/min, column temperature: 30° C., and injection volume: 2 μL.

Mass spectrometry condition: The electrospray ionization source (ESI) was used with a positive ion monitoring mode, the scanning mode was multiple reaction detection mode (MRM), and the ions used for detection were: m/z 263.1→227.2 (Compound 1), m/z 308.2→116.0 (Compound 24), and m/z 270.9→91.0 (IS). The working parameters of MS were set as follows: the capillary voltage was 1000 V, the desolventizing temperature was 600° C., and the desolventizing flow rate was 1000 L/Hr. The cone voltages of Compound 1, Compound 25 and IS were 26 V, 48 V and 14 V, respectively, and the collision energies were 44 V, 18 V and 30 V, respectively. Masslynx 4.2 was used for data collection and analysis.

### Plasma Sample Processing

After investigation by methodology, the plasma samples were pretreated by 1:3 protein precipitation method, wherein methanol was selected as the protein precipitation agent. 50 μL of rat plasma sample was aspirated, into which was added 150 μL of an internal standard methanol solution (0.67 ng/ml), and centrifuged under the condition of 14000 rpm/min and 4° C. for 10 min. The supernatant was taken, injected in 2 μL, and analyzed by LC-MS/MS.

### Pharmacokinetic Study

A total of 24 SD rats (male) were randomly divided into intragastric administration groups and tail vein injection groups (Compound 1 and Compound 23), with 6 rats in each group.

Respectively, the rats were administered intragastrically Compound 1 (0.345 mmol/kg) and Compound 23 (0.345 mmol/kg) in equal moles with 0.5% sodium carboxymethyl cellulose (containing 10% DMSO) as solvent; and injected intravenously Compound 1 (0.024 mmol/kg) and Compound 23 (0.024 mmol/kg) in equal moles with normal saline (containing 5% DMSO) as solvent. Blood samples were taken from the orbits of rats after intragastric administration at 0.167, 0.25, 0.5, 0.75, 1, 1.5, 2, 4, 5, 6, 7, 8, 10, 12 and 24 h after administration; and blood samples were taken from the orbits of rats after tail vein injection at 0.033, 0.083, 0.167, 0.25, 0.5, 0.75, 1, 1.5, 2, 4, 6, 8, 10, 12 and 24 hours after administration. The whole blood collected from orbital venous plexus was placed in 1.5 mL EP tube treated with a heparin sodium solution, and centrifuged under the condition of 8000 rpm/min and 4° C. for 10 min to obtain plasma, which was stored at −20° C. for subsequent use.

### Data Analysis

Pharmacokinetic parameters including elimination half-life (t_{1/2}), area under concentration-time curve (AUC), mean residence time (MRT), apparent distribution volume (V_{z/F}) and plasma clearance rate (CL_{z/F}) were calculated by non-compartment model of the DAS (Drugs and Statistics, Version 3.0) software. The maximum concentration (Cₘₐₓ) and the time to reach the maximum concentration (Tₘₐₓ) were determined according to the concentration-time curve. All data are expressed as mean±standard deviation (SD).

### Experimental Result

As shown in Table 3, for intragastric administration of Compound 1 and Compound 23 in equal moles, the AUC value of Compound 1 in blood after intragastric administration of Compound 23 is nearly 2 times that of Compound 1 in blood after intragastric administration of Compound 1, and the Cmax value of Compound 1 in blood after intragastric administration of Compound 23 is 10 times that of Compound 1 in blood after intragastric administration of Compound 1, which shows that the prodrug significantly improves the oral absorption of Compound 1.

TABLE 3
Main pharmacokinetic parameters of Compound 1 in rat
blood after intragastric administration of Compound
1 (0.345 mmol/kg) and Compound 23 (0.345 mmol/kg)

| | | 1 (Intragastric | 1 (Intragastric |
|---|---|---|---|
| | | administration of | administration of |
| Parameter | Unit | Compound 1) | Compound 23) |
| AUC₍₀₋ₜ₎ | ug/L*h | 350.54 ± 134.48 | 666.57 ± 103.39 |
| AUC_{(0-∞)} | ug/L*h | 351.33 ± 133.66 | 676.70 ± 103.25 |
| Cₘₐₓ | ug/L | 42.83 ± 19.73 | 458.37 ± 96.05 |
| Tₘₐₓ | h | 8.83 ± 2.04 | 0.38 ± 0.14 |
| t_{1/2} | h | 6.54 ± 0.98 | 2.74 ± 0.90 |
| MRT₍₀₋ₜ₎ | h | 9.30 ± 0.86 | 1.39 ± 0.26 |
| MRT_{(0-∞)} | h | 9.37 ± 0.76 | 1.63 ± 0.29 |

### Example 8: Activity Test of Compound for Inhibiting Activation of NLRP3 Inflammasome

NLRP3 is an important pattern recognition receptor, that can form the NLRP3 inflammasome through the adaptor ASC and pro-caspase-1. After activation, the NLRP3 inflammasome can mediate the activation of caspase-1, thus promoting the maturation and secretion of IL-1β. In order to determine whether the prepared guaianolide sesquiterpene lactone Compounds 1-8 can inhibit the activation of the NLRP3 inflammasome, we used LPS and ATP to induce the activation of the NLRP3 inflammasome, and observed the effects of Compounds 1-8 on the IL-1β level caused by NLRP3 inflammasome activation.

### Experimental Material

### Experimental Reagent

The drugs of the present application were prepared according to the above examples; Lipopolysaccharide (LPS) and Adenosine triphosphate (ATP), Sigma Company; Recombinant mouse macrophage colony stimulating factor (rmM-CSF), PeproTech Company; RPMI 1640 medium, DMEM medium and Fetal bovine serum (FBS).

### Experimental Animal

C57BL/6 mice, female, 6-8 week old and weighing 18-20 g, were provided by Qinglong Mountain Animal Farm, Jiangning District, Nanjing, with the production license number of SCXK (Su) 2017-0001.

### Experimental Method

### Isolation and Culture of Mouse Bone Marrow-Derived Macrophages (BMDMs)

C57BL/6 mice were sacrificed by cervical dislocation, and then soaked in 75% alcohol for 5-10 min. Subsequently, two hind legs of the mice were cut off with scissors. Next, the meat was removed, and the leg bones were left, which were then washed with PBS for three times. After severing the bone at both ends, a sterile syringe filled with cold serum-free RPMI 1640 medium was used to flush the bone marrow into a 15 mL centrifuge tube. Subsequently, the bone marrow was centrifuged at 1500 rpm for 5 min, and the supernatant was discarded. The residue was resuspended with 1 mL of erythrocyte lysate, blown repeatedly, and then stood for 7 min to lyse erythrocytes. After centrifugation at 1500 rpm for 5 min, the supernatant was discarded. The residue was resuspended with the RPMI 1640 medium containing 100 ng/ml of rmM-CSF, and then transferred to a 6-well culture plate for culture. After 6-7 days, the state of the cells could be observed to be a long spindle shape, indicating that they are in good condition and can be used for subsequent experiments.

### Establishment of Activation Model of NLRP3 Inflammasome

The BMDMs were seeded on 6-well cell culture plates and then treated with 100 ng/ml ultrapure LPS for 3 h. The old medium was replaced by fresh serum-free medium and treated with arglabin (1, 3, 10, 30, 60, and 120 nM) for 1 h. Subsequently, the cells were stimulated with 5 mM ATP for 45 min. The supernatants were collected into 1.5 mL EP tubes for subsequent detection of IL-1β level.

TABLE 4
Inhibitory effect of derivatives 1~9 and their
prodrugs on activation of NLRP3 inflammasome

| Derivative | IC₅₀ (nM) | Prodrug | IC₅₀ (nM) |
|---|---|---|---|
| 1 | +++ | 22 | ++ |
| | | 23 | ++ |
| 2 | +++ | 24 | ++ |
| 3 | +++ | 25 | ++ |
| 4 | +++ | 26 | ++ |
| 5 | +++ | 27 | ++ |
| 6 | +++ | 28 | + |
| 7 | +++ | 29 | ++ |
| 8 | +++ | | |
| arglabin | +++ | | |

Note:
+ stands for 80 nM < IC₅₀ < 100 nM,
++ stands for 40 nM < IC₅₀ < 80 nM, and
+++ stands for IC₅₀ < 40 nM

In BMDMs, LPS and ATP were used to induce the activation of NLRP3 inflammasome, and the effects of Compounds 1-8 and 22-29 on the protein level of IL-1β were investigated. As shown in Table 4, derivatives 1˜8 all have better inhibitory activities on IL-1β, which are close to the positive compound arglabin; and the activities of prodrugs are decreased slightly.

### Example 9: Anti-Ulcerative Colitis Activity Test of Compound 1 and its Dimethyl Fumarate Prodrug 23

### Experimental Reagent

The drugs of the present application were prepared according to the above examples; Dextran sulfate sodium (DSS), MP Biomedicals Company; Mesalazine sustained-release granules (5-aminosalicylic acid, 5-ASA), Ethypharm Pharmaceutical Company, France; Sodium carboxymethyl cellulose (CMC-Na), Xilong Chemical Plant, Shantou City, Guangdong Province; Myeloperoxidase (MPO) kit, Nanjing Institute of Bioengineering; O-toluidine, Shanghai Jingchun Biochemical Technology Co., Ltd.; Hydrogen peroxide (H₂O2) and Glacial acetic acid, Nanjing Chemical Reagent Co., Ltd.

### Experimental Animal

C57BL/6 mice, female, 6-8 weeks old and weighing 18-20 g, were provided by Qinglong Mountain Animal Farm, Jiangning District, Nanjing, with the production license number of SCXK (Su) 2017-0001. The animals were free to eat and drink, and fed with standard pellet feed at room temperature of 22±2° C. and humidity of 45±10%. After adaption for 3 days, they were used for experiments.

### Experimental Method

### Establishment of Mouse Colitis Model and Administration According to Groups

The mice were randomly divided into 7 groups, one of which was randomly selected as the normal group, and the other 6 groups were the model group, arglabin (20 nmol/kg), Compound 1 (20 and 40 nmol/kg) and Compound 23 (20 and 40 nmol/kg) groups, respectively, with 6 mice in each group. Except for the normal group, the other mice were free to drink 2.5% DSS for 7 days, which was then replaced to distilled water to be drunk freely for 3 days to establish the UC model. From the first day of the model establishment, arglabin (20 nmol/kg/d), Compound 1 (20 and 40 nmol/kg) and Compound 23 (20 and 40 nmol/kg) were administered intragastrically for 10 consecutive days. The mice in the normal group and model groups were administered intragastrically the same volume of the solvent 0.5% CMC-Na.

### Score of Disease Activity Index

The general living conditions of the mice in each group were observed and the disease activity indexs (DAI) were evaluated. The daily specific observation indicator was weight, stool property and occult blood state of mice, and then the scores of the weight loss, stool property and occult blood state were added to determine the average value, so as to obtain the DAI score of each mouse, that is, DAI=(weight loss score+stool property score+occult blood state score)/3, to evaluate the disease activity.

As shown in Table 5, the scoring criteria are as follows:

TABLE 5
Score of disease activity index

| Score | Weight loss (%) | Stool property | Bleeding in stool |
|---|---|---|---|
| 0 | none | normal | negative |
| 1 | 1-5 | — | — |
| 2 | 6-10 | soft | positive |
| 3 | 11-15 | — | — |
| 4 | >15 | diarrhea | bleeding with naked eyes |

Test method of occult blood: Using o-toluidine method, a little feces was picked up into a 24-well culture plate with a cotton swab, into which was first added 200 μL of the solution of o-toluidine glacial acetic acid, and then quickly added 200 μL of 3% hydrogen peroxide solution. Those turned blue-brown within 2 minutes were positive.

### Specimen Collection

1 h after the last administration, blood was collected from the venous plexus of fundus oculi, stood at room temperature for 2 h, and centrifuged at 3000 rpm for 20 min. The serum was aspirated, aliquoted, and frozen at −70° C. for subsequent use. After the blood collection, the mice were sacrificed by cervical dislocation, and the abdominal cavity was opened. The colon tissues were removed 1 cm away from the anus, rinsed twice with pre-cooled PBS, and frozen at −70° C. for subsequent use.

### Determination of Colon Length

The shortening of colon length is one of the main characteristics of DSS-induced UC model mice. After dissecting the abdominal cavity of the UC mice, the colon and distal ileum tissues were dissociated. The external morphological changes of the colon tissues were observed, the colon length was measured and recorded, and the photos were taken.

### Determination of MPO Activity

40 mg of colon tissues of the mice in each group was taken, into which was added 400 μL of pre-cooled normal saline to prepare tissue homogenates. After centrifugation at 4° C. and 1200 rpm for 5 min, the supernatants were collected. According to the method described in the kit instructions, various reagents were added sequentially, and finally the absorbance value of each well was determined at the wavelength of 460 nm of the microplate reader to calculate the MPO activity.

MPO activity (U/g wet weight)=(OD value of test tube−OD value of control tube)/11.3×sampling amount(g)

### Statistics

All data were expressed by Means±S.E.M., and the significances of the differences were analyzed by ANOVA. Those with significant differences by ANOVA were further compared for differences between groups by one-way ANOVA and Dunnett's test. P value less than 0.05 was considered to have significant difference.

### Experimental Result

The mice in the Normal group have normal activities and defecation, and their weights are increased slowly; and the mice given DSS show symptoms such as loose stools and semi-formed stools without sticking to anus over the days of the experiment, with reduced activities and significant body weight loss. The disease activity indexes were evaluated by observing the body weight losses, stool properties and hematochezia of the mice every day. As shown in Table 6, compared with the DSS group, Compound 23 (40 nmol/kg) significantly reduces the increased DAI score of colitis caused by DSS, and the activity of the prodrug 23 is significantly stronger than that of the parent drug 1 at the same dosage.

### Effect on DAI Score

TABLE 6
DAI score

| | | Group | | | | | |
|---|---|---|---|---|---|---|---|
| | | | 1 | 1 | 23 | 23 | Arglabin |
| Days | Normal | DSS | (20 nmol/kg) | (40 nmol/kg) | (20 nmol/kg) | (40 nmol/kg) | (20 nmol/kg) |
| 1 | 0.00 ± 0.00 | 0.00 ± 0.00 | 0.00 ± 0.00 | 0.00 ± 0.00 | 0.00 ± 0.00 | 0.00 ± 0.00 | 0.00 ± 0.00 |
| 2 | 0.00 ± 0.00 | 0.50 ± 0.17 | 0.33 ± 0.14 | 0.33 ± 0.19 | 0.27 ± 0.09 | 0.16 ± 0.10 | 0.36 ± 0.20 |
| 3 | 0.00 ± 0.00 | 0.83 ± 0.32^{##} | 0.75 ± 0.28 | 0.58 ± 0.21 | 0.63 ± 0.14 | 0.54 ± 0.11 | 0.65 ± 0.20 |
| 4 | 0.00 ± 0.00 | 1.50 ± 0.35^{##} | 1.08 ± 0.37 | 1.00 ± 0.19 | 0.96 ± 0.16 | 1.20 ± 0.14* | 1.17 ± 0.19 |
| 5 | 0.00 ± 0.00 | 2.08 ± 0.50^{##} | 1.75 ± 0.50 | 1.25 ± 0.34 | 1.02 ± 0.23* | 0.61 ± 0.23* | 1.39 ± 0.13* |
| 6 | 0.00 ± 0.00 | 2.25 ± 0.39^{##} | 2.25 ± 0.44 | 1.67 ± 0.36* | 1.56 ± 0.41* | 1.07 ± 0.25** | 1.62 ± 0.10** |
| 7 | 0.00 ± 0.00 | 3.08 ± 0.16^{##} | 2.50 ± 0.17 | 1.83 ± 0.44* | 2.28 ± 0.23* | 1.38 ± 0.25** | 2.07 ± 0.17** |
| 8 | 0.00 ± 0.00 | 3.17 ± 0.32^{##} | 2.75 ± 0.39 | 1.92 ± 0.50* | 2.46 ± 0.26** | 1.57 ± 0.28** | 2.32 ± 0.10** |
| 9 | 0.00 ± 0.00 | 2.17 ± 0.17^{##} | 1.83 ± 0.17 | 1.17 ± 0.32* | 1.31 ± 0.18** | 1.05 ± 0.25** | 1.45 ± 0.13** |
| 10 | 0.00 ± 0.00 | 1.41 ± 0.21^{##} | 1.25 ± 0.16 | 0.50 ± 0.22* | 0.80 ± 0.16** | 0.35 ± 0.16** | 0.92 ± 0.06** |

Note:
Compared with the Normal group, ^{##}P < 0.01; and compared with the DSS group, *P < 0.05, and ** P < 0.01.

### Effect on Colon Length

TABLE 7
Colon length

| Group | Number | Length (cm) |
|---|---|---|
| Normal | 6 | 7.35 ± 0.06 |
| DSS | 6 | 5.38 ± 0.35^{##} |
| 1 (20 nmol/kg/d) | 6 | 6.15 ± 0.35 |
| 1 (40 nmol/kg/d) | 6 | 6.78 ± 0.28** |
| 23 (20 nmol/kg/d) | 6 | 6.51 ± 0.25** |
| 23 (40 nmol/kg/d) | 6 | 7.26 ± 0.26** |
| arglabin (20 nmol/kg/d) | 6 | 6.50 ± 0.15** |

Note:
Compared with the Normal group,
^{##}P < 0.01; and compared with the DSS group,
**P < 0.01.

As shown in Table 7, compared with the mice in the Normal group, the colon length of the mice in the DSS group is significantly shorter. Intragastric administration of Compound 23 (40 nmol/kg) significantly inhibits the shortening of colon length, and the activity of the prodrug 23 is significantly stronger than that of the parent drug 1 at the same dosage.

### Effect on MPO Activity in Colon Tissue

The granulocytes can synthesize myeloperoxidase MPO in bone marrow before entering the blood circulation, and the latter is stored in azurophil granules. MPO can kill pathogenic microorganisms and regulate inflammatory responses by producing hypochlorous acid. MPO accounts for about 5% of dry weight of cells, and this feature can be used to determine the number of neutrophils in tissues. As shown in Table 8, compared with the mice in the Normal group, the MPO activities of the mice in the DSS group are significantly increased. Intragastric administration of 23 (40 nmol/kg) significantly reduces the MPO activities in colon tissues of mice, and the activity of prodrug 23 is significantly stronger than that of the parent drug 1 at the same dosage.

TABLE 8
MPO activity in colon tissue

| Group | Number | MPO activity (U/g tissue) |
|---|---|---|
| Normal | 6 | 0.62 ± 0.05 |
| DSS | 6 | 1.72 ± 0.28^{##} |
| 1 (20 nmol/kg) | 6 | 1.40 ± 0.31 |
| 1 (40 nmol/kg) | 6 | 1.08 ± 0.12** |
| 23 (20 nmol/kg) | 6 | 1.29 ± 0.11** |
| 23 (40 nmol/kg) | 6 | 0.99 ± 0.18** |
| arglabin (20 nmol/kg) | 6 | 0.95 ± 0.18** |

Note:
Compared with the Normal group,
^{##}P < 0.01; and compared with the DSS group,
**P < 0.01.

### Example 10: Anti-Acute Gouty Arthritis Activity Test of Compound 23

### Experimental Reagent

The drugs of the present application were prepared according to the above Example 2; Colchicine tablets, Xishuangbanna Banna Pharmaceutical Co., Ltd.; Prednisolone acetate tablets, Tianjin Xinyi Jinjin Pharmaceutical Co., Ltd.; Sodium carboxymethyl cellulose (CMC-Na), Sinopharm Chemical Reagent Co. Ltd.; Pentobarbital sodium, (China National Pharmaceutical Group) Shanghai Chemical Reagent Company; Sodium urate (MSU), Sigma-Aldrich; and IL-1β radioimmunoassay kit, IL-18 radioimmunoassay kit, Beijing Huaying Institute of Biotechnology.

### Experimental Animal

SPF SD rats, female, weighing 200-220 g, were provided by Hangzhou Medical College, with the production license number of SCXK (Zhe) 2019-0002. Animal use license number: SYXK (Su) 2019-0004. The animals were free to eat and drink, and fed with standard pellet feed at room temperature of 22±2° C. and humidity of 45±10%. After adaption for 3 days, they were used for experiments.

### Experimental Method

### Rat Acute Gouty Arthritis Model

During the adaptive feeding period, 2% pentobarbital sodium was used to anesthetize the rats by intraperitoneal injection at a dose of 0.25 mL/100 g. The circumferences of the ankle joints of the parent left hind limbs of the rats were measured. After the adaptive feeding, 2% pentobarbital sodium was used for intraperitoneal injection anesthesia at a dose of 0.25 mL/100 g. After the anesthesia, 8 rats were randomly selected as the blank group, and normal saline was injected into the joint cavity of their left hind limbs. The other rats were injected with 200 μL sodium urate into the joint cavity of the left hind limbs. 6 h after the model establishment, the rats were anesthetized with pentobarbital sodium, and the joint circumference was measured with a 2 mm wide paper strip. According to the joint swelling ratio, the rats were divided into seven groups. A blank group (equal volume of CMC-Na); a model and positive control group (equal volume of CMC-Na), a colchicine group (0.5 mg /kg), a prednisolone group (3.125 mg/kg) and a low, medium and high dose group (1.5 mg/kg, 5.0 mg/kg and 15 mg/kg) of Compound 23, with 8 rats in the blank group and 10 rats in each of the other groups.

### Administration According to Groups

Administration was carried out once at 10.5 h and 22.5 h after the model establishment, respectively. Rats in the blank group and the model group were administered intragastrically CMC-Na (0.5%), rats in the colchicine group were administered intragastrically a colchicine solution, rats in the prednisolone group were administered intragastrically a prednisolone solution, and rats in each dose group of Compound 23 were administered intragastrically Compound 23 with different concentrations, and the administration volume was 10 mL/kg.

### Evaluation of Joint Swelling Degree

Joint swelling is one of the main characteristics of acute gouty arthritis model rats induced by MSU. Before the model establishment, and at 6 h, 12 h and 24 h after the model establishment, the circumferences of the ankle joints of the left hind limbs were measured respectively, and the joint swelling ratios were calculated. The circumferences at 0.5 mm below the ankle joints of the left hind feet of rats in each group were measured with a 2 mm wide paper strip and a ruler, the measurement was repeated twice, and the average value was taken to calculate the joint swelling ratio.

Joint swelling ratio=(joint circumference at test time point−initial joint circumference)/initial joint circumference

### Statistics

All data were expressed by Means±S.E.M., and the significances of the differences were analyzed by ANOVA. Those with significant differences by ANOVA were further compared for differences between groups by one-way ANOVA and Dunnett's test. P value less than 0.05 was considered to have significant difference.

### Experimental Result

TABLE 9
Effect of Compound 23 on joint swelling ratio in rats (Mean ± SEM)

| Group | Number | 6 h | 12 h | 24 h |
|---|---|---|---|---|
| blank group | 8 | 5.31 ± 0.87 | 2.01 ± 0.25 | 2.15 ± 0.41 |
| model group | 10 | 16.41 ± 1.36^{##} | 33.09 ± 2.83^{##} | 31.43 ± 2.37^{##} |
| colchicine group | 10 | 16.15 ± 1.37 | 29.10 ± 3.17 | 23.28 ± 2.14* |
| prednisolone group | 10 | 15.78 ± 1.22 | 25.43 ± 2.03 | 18.48 ± 1.66** |
| low dose group of Compound 23 | 10 | 15.90 ± 0.91 | 30.89 ± 1.70 | 23.55 ± 2.23* |
| medium dose group of Compound 23 | 10 | 15.94 ± 1.27 | 29.69 ± 2.92 | 21.45 ± 2.01** |
| high dose group of Compound 23 | 10 | 16.00 ± 1.33 | 28.84 ± 3.401 | 19.93 ± 2.59** |

Note:
Compared with the blank group,
^{##}P < 0.01, and compared with the model group,
*P < 0.05, and
**P < 0.01.

### Effect on Joint Swelling Ratio Induced by MSU

As shown in Table 9, the swelling ratios of ankle joints of rats in the model group at 6 h, 12 h and 24 h after the model establishment are significantly higher than that in the control group; and the swelling ratios of ankle joints of rats in each administration group at 1.5 h after the first administration (12 h after the model establishment) are all decreased, but there is no significant difference. The swelling ratios of ankle joints of rats in each administration group at 1.5 h after the second administration (24 h after the model establishment) are all decreased significantly, suggesting that Compound 23 can significantly reduce the swelling ratios of ankle joints of rats with acute gouty arthritis induced by MSU.

### Example 11: Anti-Sepsis Activity Test of Compound 1, its Dimethyl Hydrochloride Prodrug 22 and Fumarate Prodrug 23 in Mice

### Experimental Reagent

The medicines of the present application were prepared according to Example 2; Lipopolysaccharide (LPS), Sigma Company; Sodium carboxymethyl cellulose (CMC-Na), Xilong Chemical Plant, Shantou City, Guangdong; RNA isolater total RNA extraction reagent, Hiscript Q RT Supermix for qPCR, Nanjing Novezan Biotechnology Co., Ltd.; and AceQ qPCR SYBR Green Master Mix, Shanghai Yisheng Biotechnology Co., Ltd.

### Experimental Animal

C57BL/6 mice, male, 6-8 weeks old and weighing 18-20 g, were provided by Shanghai Silaike Animal Centre, Jiangning District, Nanjing, with the production license number of SCXK

(Su) 2019-002. The animals were free to eat and drink, and fed with standard pellet feed at room temperature of 22±2° C. and humidity of 45±10%. After adaption for 7 days, they were used for experiments.

### Experimental Method

### Establishment of Mouse Sepsis Model and Administration According to Groups

The mice were randomly divided into 8 groups, one of which was randomly selected as the normal group, and the other 7 groups were the model group, the parent drug 1 (0.4 and 4 μmol/kg), the hydrochloride 22 (4 μmol/kg), the fumarate 23 (0.4 and 4 μmol/kg) and dexamethasone (Dex, 12 μmol/kg) groups, respectively, with 6 mice in each group. 5 days before model establishment, the parent drug 1 (0.4 and 4 μmol/kg), the hydrochloride 22 (4 μmol/kg), the fumarate 23 (0.4 and 4 μmol/kg) and Dex (12 μmol/kg) were administered intragastrically. The mice in the normal group and model group were administered intragastrically the same volume of the solvent 0.5% CMC-Na. LPS (10 mg/kg) was injected intraperitoneally 1 hour after administration on the last day to establish a mouse sepsis model.

### Specimen Collection

At 6 h after intraperitoneal injection of LPS, the mice were sacrificed by cervical dislocation, and the abdominal cavity was opened. The lungs, livers and kidneys were taken out, and rinsed twice with pre-cooled PBS. The lung tissues, liver left lobe tissues and kidney tissues of the mice were cut, and fixed with 4% paraformaldehyde for 24-48 h. The remaining tissues were frozen at −70° Cfor subsequent use.

### Q-PCR Analysis

### (1) Extraction of Total RNA

20 mg of kidney, lung and liver tissues were accurately weighed, rinsed with pre-cooled PBS solution, cut into small pieces by ophthalmic surgical scissors, and put into a glass homogenizer, into which was added 1 mL Trizol reagent for grinding to prepare tissue homogenate. Subsequently, the homogenate was transferred into a 1.5 mL EP tube without RNase and lysed on ice for 10 min. 200 μL chloroform was added, and the mixture was shaken violently, stood on ice for 3-5 min, and centrifuged at 12000 rpm and 4° C. for 15 min. The supernatant was aspirated carefully into a new 1.5 mL EP tube without RNase, into which was added the same volume of pre-cooled isopropanol, stood on an ice bath for 30 min, centrifuged at 12000 rpm and 4° C. for 10 min, and the supernatant was discarded. 75% ethanol-DEPC solution was added to wash the precipitate, and the total RNA was obtained by inversion and air drying on flat paper. Subsequently, 10 μL of DEPC treated water was added for dissolution. 1 μL of the total RNA solution was taken, and diluted to 100 times, the OD values of which at 260 nm and 280 nm were measured with a full wavelength microplate reader. If the ratio of OD260/OD280 is between 1.8 and 2.0, it is said that the extracted RNA has a high purity, i.e., it can be used in subsequent experiments.

(2) Synthesis of cDNA

Subsequent experiments were carried out with 20 μL of a reverse transcription reaction system according to the method described in the kit instructions:

| Components | Volume |
|---|---|
| 5 × qRT SuperMix II | 4 μL |
| RNase-free Water | 8 μL |
| Total RNA | 8 μL |
| Total volume | 20 μL |

The above reagents were gently blown and mixed well with a pipette, and the reaction conditions were as follows:

| Temperature | Time |
|---|---|
| 25° C. | 10 min |
| 42° C. | 30 min |
| 85° C. | 5 min |
| 4° C. | — |

cDNA obtained after the synthesis was stored at −80° C. or immediately used for the subsequent Q-PCR reaction.

### (3) Design of Primer

According to the nucleotide sequences of mice in GenBank, the sequences were designed and the primers were synthesized.

### (4) PCR Amplification

Subsequent experiments were carried out with 20 μL of a reaction system according to the method described in the kit instructions:

| Components | Volume | |
|---|---|---|
| AceQ ® qPCR SYBR ® Green Master Mix | 10 | μL |
| Forward primer (10 μM) | 0.4 | μL |
| Reverse primer (10 μM) | 0.4 | μL |
| cDNA | 2 | μL |
| RNase-free Water | 7.2 | μL |
| Total volume | 20 | μL |

The above reagents were added into a micro-reaction tube, sealed with a sealing membrane, and placed in a Q-PCR instrument. The parameters were set according to the following reaction conditions to carry out the amplification experiment. The reaction conditions were as follows:

| Stages | Temperature (° C.) | Time | Raps |
|---|---|---|---|
| Stage 1 Predegeneration | 95 | 5 min | 1 |
| | 95 | 10 sec | |
| Stage 2 Circular reaction | Tm | 30 sec | 40 |
| | 95 | 15 sec | |
| Stage 3 Melting curve | 60 | 60 sec | 1 |
| | 95 | 15 sec | |

The threshold cycle (Ct) value of each group was recorded. Calculating and analyzing were carried out by the 2^{-ΔΔCt} method (the expression level of the control gene Gapdh was set to 1 to correct the expression level of the target gene).

### Statistics

All data were expressed by Means±S.E.M., and the significances of the differences were analyzed by ANOVA. Those with significant differences by ANOVA were further compared for differences between groups by one-way ANOVA and Dunnett's test. P value less than 0.05 was considered to have significant difference.

### Experimental Result

Effect on Inflammatory Response in Lung of Mice with Sepsis

As shown in Table 10, compared with the Normal group, the mRNA expressions of inflammatory factors (Tnf, Il6, and Il1) in the lungs of mice with sepsis increased significantly, indicating that the lungs of mice with sepsis have obvious inflammatory responses. After the intervention of the parent drug 1 (4 μmol/kg), the hydrochloride 22 (4 μmol/kg) and the fumarate 23 (4 μmol/kg), the mRNA expressions of Tnf, Il6, and Il1 in lung tissues of mice are significantly down-regulated. It is suggested that Compounds 1, 22 and 23 can all inhibit the expressions of inflammatory factors and improve lung injury in mice with sepsis.

TABLE 10
mRNA expression of inflammatory factor in lung

| Group | Tnf | Il1 | Il6 |
|---|---|---|---|
| Normal | 1.00 ± 0.07 | 1.00 ± 0.04 | 1.00 ± 0.01 |
| LPS | 10.92 ± 1.57^{##} | 19.01 ± 2.40^{##} | 20.34 ± 1.96^{##} |
| 1 (0.4 μmol/kg) | 8.58 ± 0.85 | 15.29 ± 0.88 | 19.17 ± 0.51 |
| 1 (4 μmol/kg) | 7.99 ± 1.36 | 7.89 ± 1.44* | 11.67 ± 1.84* |
| 22 (4 μmol/kg) | 6.52 ± 0.29 | 5.22 ± 0.90** | 7.16 ± 0.71** |
| 23 (0.4 μmol/kg) | 8.79 ± 1.18 | 13.30 ± 0.88 | 18.40 ± 3.16 |
| 23 (4 μmol/kg) | 8.67 ± 1.51 | 6.79 ± 0.72** | 9.10 ± 1.20** |
| Dex (12 μmol/kg) | 8.99 ± 0.76 | 7.20 ± 0.36* | 9.88 ± 0.80** |

Note:
Compared with the Normal group,
^{##}P < 0.01; and compared with the LPS group,
*P < 0.05, and
**P < 0.01.

### Effect on Inflammatory Response in Kidney of Mice With Sepsis

As shown in Table 11, compared with the Normal group, the mRNA expressions of inflammatory factors (Tnf, Il6, and Il1) in the kidney of mice with sepsis increased significantly, indicating that the kidneys of mice with sepsis have obvious inflammatory responses. After the intervention of the parent drug 1 (4 μmol/kg), the hydrochloride 22 (4 μmol/kg) and the fumarate 23 (4 μmol/kg), the mRNA expressions of Tnƒ, Il6, and Il1 in kidney of mice are significantly down-regulated. It is suggested that Compounds 1, 22 and 23 can inhibit the expressions of inflammatory factors and improve kidney injury in mice with sepsis.

TABLE 11
mRNA expression of inflammatory factor in kidney

| Group | Tnf | Il1 | Il6 |
|---|---|---|---|
| Normal | 1.00 ± 0.04 | 1.00 ± 0.04 | 1.00 ± 0.03 |
| LPS | 7.30 ± 0.51^{##} | 7.99 ± 0.85^{##} | 8.80 ± 1.67^{##} |
| 1 (0.4 μmol/kg) | 7.17 ± 1.22 | 7.01 ± 0.33 | 7.55 ± 2.08 |
| 1 (4 μmol/kg) | 6.59 ± 0.18 | 3.05 ± 0.58** | 3.11 ± 0.29* |
| 22 (4 μmol/kg) | 6.20 ± 0.60 | 2.76 ± 0.59** | 2.43 ± 0.64* |
| 23 (0.4 μmol/kg) | 6.70 ± 0.80 | 6.56 ± 0.88 | 6.05 ± 0.19 |
| 23 (4 μmol/kg) | 6.04 ± 1.62 | 2.82 ± 0.33** | 2.70 ± 0.52* |
| Dex (12 μmol/kg) | 6.62 ± 0.68 | 6.05 ± 1.03 | 5.76 ± 0.72* |

Note:
Compared with the Normal group,
^{##}P < 0.01; and compared with the LPS group,
*P < 0.05, and
**P < 0.01.

### Effect on Inflammatory Response in Liver of Mice With Sepsis

As shown in Table 12, compared with the Normal group, the mRNA expressions of inflammatory factors (Tnf, Il6, and Il1) in the liver of mice with sepsis increased significantly, indicating that the liver of mice with sepsis have obvious inflammatory responses. After the intervention of the parent drug 1 (4 μmol/kg), the hydrochloride 22 (4 μmol/kg) and the fumarate 23 (4 μmol/kg), the mRNA expressions of Tnf, Il6, and Il1 in liver tissues of mice are significantly down-regulated. It is suggested that Compounds 1, 22 and 23 can inhibit the expressions of inflammatory factors and improve liver injury in mice with sepsis.

TABLE 12
mRNA expression of inflammatory factor in lung

| Group | Tnf | Il1 | Il6 |
|---|---|---|---|
| Normal | 1.00 ± 0.01 | 1.00 ± 0.11 | 1.00 ± 0.08 |
| LPS | 8.92 ± 1.93^{##} | 11.52 ± 1.19^{##} | 11.34 ± 0.75^{##} |
| 1 (0.4 μmol/kg) | 8.75 ± 2.25 | 11.24 ± 2.98 | 8.86 ± 3.11 |
| 1 (4 μmol/kg) | 7.09 ± 0.99 | 4.82 ± 0.58** | 5.20 ± 0.87** |
| 22 (4 μmol/kg) | 5.71 ± 0.61 | 3.07 ± 0.29** | 3.93 ± 0.90** |
| 23 (0.4 μmol/kg) | 7.63 ± 1.97 | 10.46 ± 1.55 | 9.13 ± 0.80 |
| 23 (4 μmol/kg) | 7.10 ± 1.09 | 4.36 ± 0.94** | 4.97 ± 0.77** |
| Dex (12 μmol/kg) | 7.04 ± 0.77 | 7.54 ± 0.70* | 4.27 ± 1.33* |

Note:
Compared with the Normal group,
^{##}P < 0.01; and compared with the LPS group,
**P < 0.01.

### Example 12: Anti-Acute Lung Injury Activity Test of Compound 1, its Dimethyl Hydrochloride Prodrug 22 and Fumarate Prodrug 23

### Experimental Reagent

The medicines used were prepared according to Example 2; LPS was purchased from Sigma Company in the United States; Dexamethasone (Dex) was purchased from Sigma Company in the United States; and the ELISA kit was purchased from Dakewei Biological Co., Ltd.

### Experimental Animal

Male C57BL/6 mice, 6-8 weeks old and weighing 18-20 g, were purchased from Jiangsu Jicui Yaokang Technology Co., Ltd. [Production license number: SCXK (Su) 2018-0008]. The animals were free to eat and drink, and kept in an environment with room temperature of 22±2° C. and humidity of 45±10%. After accommodating feeding for 7 days, they were used for subsequent experiments.

### Experimental Method

### Establishment of Mouse Acute Lung Injury Model and Administration According to Groups

Treatment of acute lung injury by intragastric injection of 1, 22 and 23: Male C57BL/6J mice, 6-8 weeks old and weighing 18-20 g, were randomly divided according to their weights into the normal group, the model group, 1, 22, 23 (0.4 and 4 μmol/kg) group and the positive control drug Dex (12 μmol/kg) group, with 8 mice in each group. In addition to the normal group injected intragastrically with PBS, other mice were injected intraperitoneally with LPS (7.5 mg/kg) to establish acute lung injury models. Three days before model establishment, the parent drug 1 (0.4 and 4 μmol/kg), the hydrochloride 22 (0.4 and 4 μmol/kg), the fumarate 23 (0.4 and 4 μmol/kg) and the positive drug Dex (12 μmol/kg) were administered intragastrically in advance, and LPS (7.5 mg/kg) was injected intraperitoneally half an hour after administration on the third day. Lung tissues were collected 12 hours after model establishment, and the effects of 1, 22 and 23 on the expressions of inflammatory factors in lung tissues of mice with acute lung injury were detected by ELISA (Table 6).

### Specimen Collection

12 h after last administration, the mice were sacrificed by cervical dislocation, and the

abdominal cavity was opened. The lungs were taken out, and rinsed twice with pre-cooled PBS. The lung tissues of the mice were cut, and fixed with 4% paraformaldehyde for 24-48 h. The remaining tissues were frozen at −70° C. for subsequent use.

### ELISA Analysis

### Extraction of Total Protein

20 mg of lung tissues were accurately weighed, rinsed with pre-cooled PBS solution, cut into small pieces by ophthalmic surgical scissors, and put into a glass homogenizer, into which was added 180 μL PBS (containing 1 mM PMSF). The mixture was homogenized at 60 HZ for 180 s, and centrifuged at 3000 rpm/min at 4° C. for 20 min. 1 μL supernatant was taken, the protein concentration of which was measured with Nanodrop and leveled, and could be used for subsequent experiments.

### Statistics

All the data were expressed by means±S.E.M., and the statistical differences between groups were tested by one-way ANONA and t test in the SPSS software. P value less than 0.05 was considered to have significant difference.

### Experimental Result

### Effect of Treatment by Intragastric Injection and Administration of 1, 22 and 23 on Acute Lung Injury in Mice

As shown in Table 13, compared with the Normal group, the protein expressions of inflammatory factors (IL-1β and IL-6) in the lung tissues of mice are increased significantly, indicating that the lungs of mice have obvious inflammatory responses. After the intervention of the parent drug 1 (0.4 and 4 μmol/kg), the hydrochloride 22 (0.4 and 4 μmol/kg) and the fumarate 23 (0.4 and 4 μmol/kg), the protein expressions of IL-1β and IL-6 in lung tissues of mice are significantly down-regulated. It is suggested that Compounds 1, 22 and 23 can inhibit the expressions of inflammatory factors and improve acute lung injury in mice.

TABLE 13
Expression of inflammatory factor (IL-1β and
IL-6) in serum of mice treated with intraperitoneal
injection and administration of 1, 22 and 23

| Group | Number | IL-1β (pg/mL) | IL-6 (pg/mL) |
|---|---|---|---|
| Normal | 8 | 88.94 ± 0.91 | 71.14 ± 9.55 |
| LPS | 8 | 2411.32 ± 48.50 | 3520.94 ± 222.10 |
| 1 (0.4 μmol/kg) | 8 | 1824.88 ± 32.73 | 2760.57 ± 288.02 |
| 22 (0.4 μmol/kg) | 8 | 1570.02 ± 62.95* | 2348.32 ± 202.75 |
| 23 (0.4 μmol/kg) | 8 | 1623.55 ± 32.64* | 2585.26 ± 273.50 |
| 1 (4 μmol/kg) | 8 | 945.66 ± 36.71 | 1314.91 ± 231.08 |
| 22 (4 μmol/kg) | 8 | 708.74 ± 38.40* | 1172.00 ± 240.50 |
| 23 (4 μmol/kg) | 8 | 888.28 ± 35.20* | 1288.32 ± 123.80** |
| Dex (12 μmol/kg) | 8 | 1873.00 ± 86.52 | 1423.00 ± 168.40*** |

Note:
*P < 0.05,
**P < 0.01, and
***P < 0.001 (compared with the LPS group)

### Example 13: Anti-Liver Injury Activity Test of Compound 1, its Dimethyl Hydrochloride Prodrug 22 and Fumarate Prodrug 23

### Experimental Reagent

The medicines of the present application were prepared according to Example 2; Olive oil, Beijing Cleopatra Olive Oil Development Center; Analytical pure carbon tetrachloride (CCl₄), Shantou Xilong Chemical Plant; Sodium carboxymethyl cellulose (CMC-Na), Xilong Chemical Plant, Shantou City, Guangdong; RNA isolater total RNA extraction reagent, Hiscript Q RT Supermix for qPCR, Nanjing Novezan Biotechnology Co., Ltd.; AceQ qPCR SYBR Green Master Mix, Shanghai Yisheng Biotechnology Co., Ltd.; and Aspartate transaminase/glutamic oxalacetic transaminase (AST/GOT) colorimetric test kit, and Alanine transaminase/glutamic-pyruvic transaminase(ALT/GPT) colorimetric test kit, Wuhan Elabscience Biotechnology Co., Ltd.

### Experimental Animal

C57BL/6 mice, male, 6-8 weeks old and weighing 18-20 g, were provided by Shanghai Silaike Animal Centre, Jiangning District, Nanjing, with the production license number of SCXK (Su) 2019-002. The animals were free to eat and drink, and fed with standard pellet feed at room temperature of 22±2° C. and humidity of 45±10%. After adaption for 7 days, they were used for experiments.

### Experimental Method

### Establishment of Mouse Acute Liver Injury Model and Administration According to Groups

The mice were randomly divided into 8 groups, one of which was randomly selected as the normal group, and the other 7 groups were the model group, the parent drug 1 (0.4 and 4 μmol/kg), the hydrochloride 22 (4 μmol/kg), and the fumarate 23 (0.4 and 4 μmol/kg) groups, respectively, with 6 mice in each group. In addition to the normal group injected with 10 mL olive oil/kg, the other mice were injected intraperitoneally with 0.5 mL CCl₄+9.5 mL olive oil/kg weight, i.e., the ratio of CCl₄ to olive oil was 1:19. From the first day of model establishment, the parent drug 1 (0.4 and 4 μmol/kg), the hydrochloride 22 (4 μmol/kg) and the fumarate 23 (0.4 and 4 μmol/kg) were administered intragastrically, respectively, for 3 consecutive days. The mice in the normal group and model group were administered intragastrically the same volume of the solvent 0.5% CMC-Na.

### Specimen Collection

1 h after the last administration, blood was collected from the venous plexus of fundus oculi, stood at room temperature for 2 h, and centrifuged at 3000 rpm for 20 min. The serum was aspirated, aliquoted, and frozen at −70° C. for subsequent use. After blood collection, the mice were sacrificed by cervical dislocation, and the abdominal cavity was opened. The livers were taken out, and rinsed twice with pre-cooled PBS. The liver left lobe tissues of the mice were cut, and fixed with 4% paraformaldehyde for 24-48 h. The remaining tissues were frozen at −70° C. for subsequent use.

### Determination of AST and ALT in Serum

μL of each sample was taken for experiments. The levels of AST and ALT in the serum of mice were detected according to the operation manual. The absorbance value was detected at the wavelength of 510 nm by a microplate reader, and the sample concentration (U/L) was calculated according to the standard curve.

### Q-PCR Analysis

The extraction method of total RNA from tissues, cDNA synthesis and the PCR amplification method are the same as those in Example 4.

### Statistics

All data were expressed by Means±S.E.M., and the significances of the differences were analyzed by ANOVA. Those with significant differences by ANOVA were further compared for differences between groups by one-way ANOVA and Dunnett's test. P value less than 0.05 was considered to have significant difference.

### Experimental Result

### Effect on ALT and AST Levels in Serum

The changes of ALT and AST levels in serum are important indicators to measure liver injury. In order to determine whether CCl₄ really induced acute liver injury in mice, the ALT and AST levels in serums of the model group mice were first detected in this study. The results show that compared with the normal olive oil group, the ALT and AST levels in serums of mice treated with CCl4 increase significantly. It is suggested that the mouse acute liver injury model is established successfully. In addition, intragastric administration of the parent drug 1 (4 μmol/kg), the hydrochloride 22 (4 μmol/kg) and the fumarate 23 (4 μmol/kg) significantly decreases the ALT and AST levels in serums of mice. Therefore, Compounds 1, 22 and 23 can improve liver injury in mice.

TABLE 14
ALT and AST levels

| Group | ALT | | AST | |
|---|---|---|---|---|
| Normal | 9.47 | ± 3.21 | 41.42 | ± 24.62 |
| CCl₄ | 171.46 | ± 3.42^{##} | 430.64 | ± 54.91^{##} |
| 1 (0.4 μmol/kg) | 174.83 | ± 22.05 | 425.20 | ± 178.43 |
| 1 (4 μmol/kg) | 105.14 | ± 5.39** | 239.08 | ± 39.59* |
| 22 (4 μmol/kg) | 85.02 | ± 15.84** | 153.34 | ± 27.59** |
| 23 (0.4 μmol/kg | 134.21 | ± 34.25 | 387.71 | ± 29.17 |
| 23 (4 μmol/kg) | 99.46 | ± 2.69** | 216.14 | ± 65.51* |
| Silybin | 121.58 | ± 15.45* | 301.45 | ± 38.44* |
| (400 μmol/kg) | | | | |

Note:
Compared with the Normal group, ^{##}P < 0.01; and compared with the CCl₄ group, *P < 0.05, and **P < 0.01.

Effect on mRNA Expression of Fibrosis-Related Factor in Liver Tissue

The effects of the parent drug 1, the hydrochloride 22 and the fumarate 23 on proinflammatory factors in livers of mice were evaluated by detecting the changes of mRNA expression levels of fibrosis-related factors (Acta2, Colla1, and Tgfb1) in liver tissues of mice in each group. As shown in Table 15, compared with the Normal group, the mRNA levels of Acta2, Colla1 and Tgfb1 in liver tissues are significantly increased at 72 h after intraperitoneal injection of CCl₄, and their expressions are significantly decreased in treatment groups of the parent drug 1 (4 μmol/kg), the hydrochloride 22 (4 μmol/kg) and the fumarate 23 (4 μmol/kg). Therefore, Compounds 1, 22 and 23 can inhibit fibrosis reactions in liver induced by CCl₄ to some extent.

TABLE 15
mRNA expression of fibrosis-related factor in liver

| Group | Acta2 | | Col1a1 | | Tgfb1 | |
|---|---|---|---|---|---|---|
| Normal | 1.00 | ± 0.00 | 1.00 | ± 0.00 | 1.00 | = 0.00 |
| CCl₄ | 4.35 | ± 0.71^{##} | 2.75 | ± 0.25^{##} | 1.66 | ± 0.12^{##} |
| 1 (0.4 μmol/kg) | 3.78 | ± 0.90 | 2.33 | ± 0.87 | 1.43 | ± 0.71 |
| 1 (4 μmol/kg) | 2.41 | ± 0.18* | 1.42 | ± 0.29** | 1.12 | ± 0.19** |
| 22 (4 μmol/kg) | 1.85 | ± 0.77** | 1.16 | ± 0.26** | 0.92 | ± 0.21** |
| 23 (0.4 μmol/kg) | 2.97 | ± 0.26 | 1.83 | ± 0.53 | 1.08 | ± 0.29** |
| 23 (4 μmol/kg) | 2.15 | ± 0.44* | 1.33 | ± 0.31** | 0.97 | ± 0.13** |
| Silybin | 2.91 | ± 0.48* | 1.84 | ± 0.17* | 1.11 | ± 0.08* |
| (400 μmol/kg) | | | | | | |

Note:
Compared with the Normal group, ^{##}P < 0.01; and compared with the CCl₄ group, *P < 0.05, and **P < 0.01.

### Example 14: Effect of Compound 1, its Dimethyl Hydrochloride Prodrug 22 and Fumarate Prodrug 23 on Kidney Injury in Mice Induced by Folic Acid and Unilateral Ureteral Obstruction (UUO)

### Experimental Reagent

The medicines of the present application were prepared according to Example 2; Folic acid (FA), MP Biomedicals Company; Sodium carboxymethyl cellulose (CMC-Na), Xilong Chemical Plant, Shantou City, Guangdong; RNA isolater total RNA extraction reagent, Hiscript Q RT Supermix for qPCR, Nanjing Novezan Biotechnology Co., Ltd.; and AceQ qPCR SYBR Green Master Mix, Shanghai Yisheng Biotechnology Co., Ltd.; and Urea colorimetry test kit, and Creatinine colorimetry test kit, Wuhan Elabscience Biotechnology Co., Ltd.

### Experimental Animal

C57BL/6 mice, male, 6-8 weeks old and weighing 18-20 g, were provided by Shanghai Silaike Animal Centre, Jiangning District, Nanjing, with the production license number of SCXK (Su) 2019-002. The animals were free to eat and drink, and fed with standard pellet feed at room temperature of 22±2° C. and humidity of 45±10%. After adaption for 7 days, they were used for experiments.

### Experimental Method

Establishment of Mouse Kidney Injury Model and Administration According toGroups

Acute kidney injury: The mice were randomly divided into 7 groups, one of which was randomly selected as the normal group, and the other 6 groups were the model group, the parent drug 1 (0.4 and 4 μmol/kg), the hydrochloride 22 (4 μmol/kg), and the fumarate 23 (0.4 and 4 μmol/kg) groups, respectively, with 6 mice in each group. In addition to the mice in the normal group, other mice were injected intraperitoneally with folic acid dissolved in 0.9% NaHCO₃ solution (250 mg/kg) to establish mouse acute kidney injury models. 1 h before model establishment, the parent drug 1 (0.4 and 4 μmol/kg), the hydrochloride 22 (4 μmol/kg) and the fumarate 23 (0.4 and 4 μmol/kg) were administered intragastrically for 3 consecutive days. The mice in the normal group and model group were administered intragastrically the same volume of the solvent 0.5% CMC-Na.

Chronic kidney injury: The mice were randomly divided into 7 groups, one of which was randomly selected as the normal group, and the other 6 groups were the model group, the parent drug 1 (0.4 and 4 μmol/kg), the hydrochloride 22 (4 μmol/kg), and the fumarate 23 (0.4 and 4 μmol/kg) groups, respectively, with 6 mice in each group. In addition to mice in the normal group that had sham operations (i.e., there was no other operations, but only the left ureter was exposed), the other mice were established chronic kidney injury models by the unilateral ureteral obstruction method. Specifically, the abdominal skin was sterilized with iodophor and alcohol in sequence, and the abdominal skin was cut and separated layer by layer. After fully exposing and separating the left ureter, double obstruction was performed at the proximal bladder end of the ureter with a digestible suture line, followed by suture. On the first day of model establishment, the parent drug 1 (0.4 and 4 μmol/kg), the hydrochloride 22 (4 μmol/kg) and the fumarate 23 (0.4 and 4 μmol/kg) were administered intragastrically for 14 consecutive days. The mice in the normal group and model group were administered intragastrically the same volume of the solvent 0.5% CMC-Na.

### Specimen Collection

1 h after the last administration, blood was collected from the venous plexus of fundus oculi, stood at room temperature for 2 h, and centrifuged at 3000 rpm for 20 min. The serum was aspirated, aliquoted, and frozen at −70° C. for subsequent use. After blood collection, the mice were sacrificed by cervical dislocation, and the abdominal cavity was opened. The kidneys were taken out, rinsed twice with pre-cooled PBS after the perirenal fats and capsules were peeled off, and then fixed with 4% paraformaldehyde for 24-48 h, embedded in paraffin and stained with HE. The remaining tissues were frozen at −70° C. for subsequent use.

### Determination of Serum Creatinine (Scr) and Urea (BUN) Levels in Blood

μL serum was taken from each sample for the detection of creatinine and urea levels. Samples were added in accordance with the instructions of the kit strictly. After incubation at 37° C., the absorbance value was measured in a microplate reader, and the content levels of detection indicators in each group were calculated according to the standard curve.

### Q-PCR Analysis

The extraction of total RNA from animal tissues, cDNA synthesis and PCR amplification are as in above examples.

### Experimental Result

### Effect on Creatinine and Urea Levels in Serum

As shown in Table 16 and Table 17, compared with the mice in the Normal group, the levels of urea and creatinine in serums of mice are significantly increased at 48 h after folic acid administration, while the levels of urea and creatinine in treatment groups of the parent drug 1 (4 μmol/kg), the hydrochloride 22 (4 μmol/kg) and the fumarate 23 (4 μmol/kg) are significantly lower than those in the model group, with statistical significance (P <0.05). It is suggested that Compounds 1, 22 and 23 have good protective effects on acute and chronic kidney injury induced by folic acid or unilateral ureteral obstruction.

TABLE 16
Creatinine and urea levels in serum-acute model

| Group | Scr | | BUN | |
|---|---|---|---|---|
| Normal | 44.90 | ± 5.63 | 7.07 | ± 0.76 |
| FA | 124.49 | ± 12.85^{##} | 33.18 | ± 2.54^{##} |
| 1 (0.4 μmol/kg) | 108.48 | ± 11.76 | 31.46 | ± 2.54 |
| 1 (4 μmol/kg) | 71.95 | ± 5.74** | 21.33 | ± 2.21** |
| 22 (4 μmol/kg) | 64.94 | ± 9.68** | 16.74 | ± 1.42 |
| 23 (0.4 μmol/kg) | 90.69 | ± 12.83 | 30.08 | ± 3.03 |
| 23 (4 μmol/kg) | 50.39 | ± 7.65** | 18.95 | ± 2.58** |

Note:
Compared with the Normal group, ^{##}P < 0.01; and compared with the FA group, *P < 0.05, and **P < 0.01.

TABLE 17
Creatinine and urea levels in serum-chronic model

| Group | Scr | | BUN | |
|---|---|---|---|---|
| Normal | 37.07 | ± 7.66 | 7.60 | ± 0.98 |
| UUO | 201.57 | ± 33.25^{##} | 20.36 | ± 1.44^{##} |
| 1 (0.4 μmol/kg) | 176.35 | ± 17.11 | 18.41 | ± 1.82 |
| 1 (4 μmol/kg) | 104.65 | ± 16.26* | 11.01 | ± 1.54* |
| 22 (4 μmol/kg) | 64.50 | ± 7.13** | 9.39 | ± 0.92** |
| 23 (0.4 μmol/kg) | 157.79 | ± 21.92 | 17.63 | ± 1.13 |
| 23 (4 μmol/kg) | 79.87 | ± 13.44** | 9.45 | ± 1.55** |

Note:
Compared with the Normal group, ^{##}P < 0.01; and compared with the UUO group, *P < 0.05, and **P < 0.01.

Effect on mRNA Expression of Kidney Injury Marker

The effects of the parent drug 1, the hydrochloride 22 and the fumarate 23 on kidney function in mice were evaluated by detecting the changes of mRNA expression levels of kidney injury markers (Ngal and Havcr1) in kidney tissues of mice in each group. As shown in Table 18 and Table 19, compared with the Normal group, the mRNA levels of Ngal and Havcrl in kidney 5 tissues of mice are significantly increased at 48 h after folic acid administration or after unilateral ureteral obstruction, and the mRNA levels of Ngal and Havcrl in treatment groups of the parent drug 1 (4 μmol/kg), the hydrochloride 22 (4 μmol/kg) and the fumarate 23 (4 μmol/kg) are significantly decreased. It is suggested that Compounds 1, 22 and 23 can inhibit the increase of kidney injury markers caused by folic acid or unilateral ureteral obstruction.

TABLE 18
mRNA expression of kidney injury marker in kidney-acute model

| Group | Havcr1 | | Ngal | |
|---|---|---|---|---|
| Normal | 1.00 | ± 0.05 | 1.00 | ± 0.04 |
| FA | 199.26 | ± 39.19^{##} | 36.25 | ± 6.78^{##} |
| 1 (0.4 μmol/kg) | 175.10 | ± 17.63 | 30.52 | ± 5.05 |
| 1 (4 μmol/kg) | 99.99 | ± 7.98* | 20.17 | ± 2.21* |
| 22 (4 μmol/kg) | 62.86 | ± 12.20 | 13.05 | ± 1.82** |
| 23 (0.4 μmol/kg) | 170.08 | ± 33.10 | 25.75 | ± 2.80 |
| 23 (4 μmol/kg) | 83.11 | ± 16.85* | 13.08 | ± 2.41** |

Note:
Compared with the Normal group, ^{##}P < 0.01; and compared with the FA group, *P < 0.05, and **P < 0.01.

TABLE 19
mRNA expression of kidney injury marker in kidney-chronic model

| Group | Havcr1 | | Ngal | |
|---|---|---|---|---|
| Normal | 1.00 | ± 0.04 | 1.00 | ± 0.05 |
| UUO | 14.49 | ± 2.23^{##} | 45.13 | ± 7.89^{##} |
| 1 (0.4 μmol/kg) | 12.56 | ± 1.95 | 37.82 | ± 4.85 |
| 1 (4 μmol/kg) | 8.54 | ± 1.38* | 21.66 | ± 6.00* |
| 22 (4 μmol/kg) | 6.29 | ± 2.43** | 16.14 | ± 2.74** |
| 23 (0.4 μmol/kg) | 11.90 | ± 4.52 | 35.77 | ± 7.67 |
| 23 (4 μmol/kg) | 7.25 | ± 0.80* | 19.42 | ± 1.55** |

Note:
Compared with the Normal group, ^{##}P < 0.01; and compared with the UUO group, *P < 0.05, and **P < 0.01.

Effect on mRNA Expression of Inflammatory Factor in Kidney Tissue

The effects of the parent drug 1, the hydrochloride 22 and the fumarate 23 on proinflammatory factors in kidneys of mice were evaluated by detecting the changes of mRNA expression levels of inflammatory factors (Tnƒ, Il6, and Il1) in kidney tissues of mice in each group. As shown in Table 13 and Table 14, compared with the Normal group, the mRNA levels of Tnƒ, Il6, and Il1 in kidney tissues of mice are significantly increased at 48 h after folic acid administration or after unilateral ureteral obstruction, and their expressions are significantly decreased in treatment groups of the parent drug 1 (4 μmol/kg), the fumarate 23 (4 μmol/kg) and the hydrochloride 22 (4 μmol/kg). Notably, the activity of the hydrochloride 22 isstronger than that of the parent drug 1 and the fumarate 23 at the same dose. It is suggested that Compound 22 can reduce inflammatory responses in kidney induced by folic acid or unilateral ureteral obstruction to some extent.

TABLE 20
mRNA expression of inflammatory factor in kidney-acute model

| Group | Tnf | | Il1 | | Il6 | |
|---|---|---|---|---|---|---|
| Normal | 1.00 | ± 0.06 | 1.00 | ± 0.05 | 1.00 | ± 0.02 |
| FA | 3.10 | ± 0.56^{##} | 2.94 | ± 0.35^{##} | 8.87 | ± 0.91^{##} |
| 1 (0.4 μmol/kg) | 2.89 | ± 0.59 | 2.62 | ± 0.56 | 8.02 | ± 0.77 |
| 1 (4 μmol/kg) | 2.76 | ± 0.71 | 1.72 | ± 0.45* | 3.98 | ± 0.59** |
| 22 (4 μmol/kg) | 2.31 | ± 0.72 | 1.06 | ± 0.33** | 1.55 | ± 0.28** |
| 23 (0.4 μmol/kg) | 2.60 | ± 0.62 | 2.07 | ± 0.38 | 6.47 | ± 0.53* |
| 23 (4 μmol/kg) | 2.42 | ± 0.35 | 1.45 | ± 0.23** | 5.61 | ± 2.10** |

Note:
Compared with the Normal group, ^{##}P < 0.01; and compared with the FA group, *P < 0.05, and **P < 0.01.

TABLE 21
mRNA expression of inflammatory factor in kidney-chronic model

| Group | Tnf | | Il1 | | Il6 | |
|---|---|---|---|---|---|---|
| Normal | 1.00 | ± 0.02 | 1.00 | ± 0.02 | 1.00 | ± 0.12 |
| UUO | 7.91 | ± 1.28^{##} | 7.98 | ± 1.04^{##} | 27.27 | ± 5.53^{##} |
| 1 (0.4 μmol/kg) | 6.41 | ± 1.46 | 6.05 | ± 0.49 | 16.52 | ± 1.69 |
| 1 (4 μmol/kg) | 6.59 | ± 1.19 | 3.99 | ± 0.31** | 6.50 | ± 1.51** |
| 22 (4 μmol/kg | 5.09 | ± 0.66 | 2.77 | ± 0.40* | 3.69 | ± 1.26** |
| 23 (0.4 μmol/kg) | 7.10 | ± 1.93 | 5.32 | ± 0.56* | 14.31 | ± 6.01* |
| 23 (4 μmol/kg) | 7.53 | ± 0.68 | 3.55 | ± 0.50** | 5.61 | ± 2.10** |

Note:
Compared with the Normal group, ^{##}P < 0.01; and compared with the UUO group, *P < 0.05, and **P < 0.01.

Effect on mRNA Expression of Fibrosis-Related Factor in Kidney Tissue

The effects of the parent drug 1, the hydrochloride 22 and the fumarate 23 on fibrosis in kidneys of mice were evaluated by detecting the changes of mRNA expression levels of fibrosis-related factors (α-Sma, Colla1l, and Tgfb1) in kidney tissues of mice in each group. As shown in Table 22 and Table 23, compared with the Normal group, the mRNA levels of Acta2, Colla1, and Tgfb1 in kidney tissues of mice are significantly increased at 48 h after folic acid administration or after unilateral ureteral obstruction, and their expressions are significantly decreased in treatment groups of the parent drug 1 (4 μmol/kg), the hydrochloride 22 (4 μmol/kg) and the fumarate 23 (4 μmol/kg). It is suggested that Compounds 1, 22 and 23 can inhibit fibrosis in kidney induced by folic acid to some extent.

TABLE 22
mRNA expression of fibrosis-related factor in kidney- acute model

| Group | α-Sma | | Col1a1 | | Tgfb1 | |
|---|---|---|---|---|---|---|
| Normal | 1.00 | ± 0.06 | 1.00 | ± 0.04 | 1.00 | ± 0.03 |
| FA | 2.16 | ± 0.26^{##} | 1.95 | ± 0.11^{##} | 2.11 | ± 0.29^{##} |
| 1 (0.4 μmol/kg) | 1.82 | ± 0.19 | 1.64 | ± 0.16 | 1.96 | ± 0.17 |
| 1 (4 μmol/kg) | 1.42 | ± 0.11* | 1.32 | ± 0.15** | 1.36 | ± 0.10* |
| 22 (4 μmol/kg) | 1.11 | ± 0.17** | 1.18 | ± 0.15** | 0.98 | ± 0.27** |
| 23 (0.4 μmol/kg) | 1.80 | ± 0.42 | 1.58 | ± 0.35 | 1.64 | ± 0.16 |
| 23 (4 μmol/kg) | 0.90 | ± 0.21** | 1.20 | ± 0.19** | 1.09 | ± 0.15* |

Note:
Compared with the Normal group, ^{##}P < 0.01; and compared with the FA group, *P < 0.05, and **P < 0.01.

TABLE 23
mRNA expression of fibrosis-related factor in kidney- chronic model

| Group | α-Sma | | Col1a1 | | Tgfb1 | |
|---|---|---|---|---|---|---|
| Normal | 1.00 | ± 0.04 | 1.00 | ± 0.01 | 1.00 | ± 0.02 |
| UUO | 2.37 | ± 0.26^{##} | 16.51 | ± 1.71^{#} | 2.12 | ± 0.16^{##} |
| 1 (0.4 μmol/kg) | 2.32 | ± 0.54 | 16.09 | ± 3.57 | 2.04 | ± 0.24 |
| 1 (4 μmol/kg) | 1.54 | ± 0.12* | 9.45 | ± 1.03* | 1.55 | ± 0.19* |
| 22 (4 μmol/kg) | 1.26 | ± 0.18** | 7.30 | ± 0.90** | 1.44 | ± 0.21* |
| 23 (0.4 μmol/kg) | 2.02 | ± 0.39 | 11.79 | ± 1.18* | 1.76 | ± 0.05 |
| 23 (4 μmol/kg) | 1.39 | ± 0.04** | 8.27 | ± 1.15** | 1.53 | ± 0.14* |

Note:
Compared with the Normal group, ^{##}P < 0.01; and compared with the UUO group, *P < 0.05, and ** P < 0.01.

### Example 15: Anti-Tumor Effect of Compound 23 in Hepa1-6 Subcutaneous Mouse Xenograft Models

### Experimental Method

Hepa1-6 cells were cultured and amplified in vitro. Appropriate amount of the cells in logarithmic growth phase were resuspended in a suspension of a serum-free DMEM medium and Matrigel (1:1) to prepare a cell suspension in 2.5×10⁶/100 μL under sterile conditions. 100 μL of the cell suspension was subcutaneously inoculated into the armpits of the front left limbs of C57BL/6 mice with a syringe. When the tumor volume grew to 100-200 mm³, animals with moderate tumor size were randomly divided into groups, with 6 animals in each group. The groups were the solvent control group, the arglabin (10 mg/kg/d) group and the 23 (8.43 mg/kg/d) group with the same molar dose as arglabin, respectively. The mice were administered intragastrically every day for 2 weeks. During the administration, the weights and tumor diameters of the mice were measured every day. After the experiment, the mice were sacrificed by cervical dislocation, and the tumors were taken and weighed. The formula for calculating a tumor volume (TV) is: TV=½×a×b², where a represents the long diameter of a tumor; b represents the short diameter of a tumor.

### Experimental Result

As shown in Table 24, in the Hepal1-6 mouse subcutaneous mouse xenograft models , after 2 weeks of continuous administration, similar to the positive compound arglabin, Compound 23 has significant inhibitory effect on tumor growth.

TABLE 24
Anti-tumor effect of Compound 23 in Hepa1-6 mouse xenograft models

| | | Number of | | | |
|---|---|---|---|---|---|
| | Dose | animals | Tumor | | Tumor growth |
| Group | (mg/kg/d) | start/end | weight (g) | | inhibition (%) |
| solvent | — | 6/6 | 0.92 | ± 1.41 | — |
| control | | | | | |
| 23 | 8.43 | 6/6 | 0.03 | ± 0.02*** | 96.78%*** |
| arglabin | 10 | 6/6 | 0.04 | ± 0.03*** | 95.86%*** |

*p < 0.05; **p < 0.01; and ***p < 0.001 (compared with the solvent control).

### Example 16: Anti-Tumor Effect of Compound 23 in H22 Subcutaneous Mouse Xenograft Models

### Experimental Method

H22 cells were cultured and amplified in vitro. Appropriate amount of the cells in logarithmic growth phase were resuspended in a suspension of a serum-free RPMI 1640 medium and Matrigel (1:1) to prepare a cell suspension in 3×105/100 μL under sterile conditions. 100 μL of the cell suspension was subcutaneously inoculated into the armpits of the front left limbs of BALB/c mice with a syringe. When the tumor volume grew to 100-200 mm³, animals with moderate tumor size were randomly divided into groups, with 6 animals in each group. The groups were the solvent control group, the arglabin (10 mg/kg/d) group, and the group with the same molar dose as arglabin, i.e., the 23 (8.43 mg/kg/d) group, respectively. The mice were administered intragastrically every day for 2 weeks. During the administration, the weights and tumor diameters of the mice were measured every day. After the experiment, the mice were sacrificed by cervical dislocation, and the tumors were taken and weighed. The formula for calculating a tumor volume (TV) is: TV=½×a×b², where a represents the long diameter of a tumor; b represents the short diameter of a tumor.

The tumor-bearing mice were sacrificed by the cervical dislocation method, placed in a beaker containing 75% alcohol, and the tumors of the mice were peeled off. The tumors were first soaked in a sterile petri dish containing RPMI 1640. After all the tumors were peeled off, fingernail sized tumor was cut off and transferred to a 1.5 mL EP tube. The tumors were cut into small pieces, and transferred into a 15 mL centrifuge tube containing 3-5 mL of digestive enzymes (Collagenase IV 1 mg/mL, Hyaluronidase 1 mg/mL, and DNase I 20 U/mL), and digested in a shaker at 200 rpm and 37° C. for 1 h. After digestion, the mixture was allowed to pass through a 200-mesh screen to obtain the suspension of the tumor cells. The resulting tumor suspension was centrifuged at 1600 rpm for 5 min. The supernatant was aspirated away. 1 mL of 1×PBS was added into each tube to resuspend the cells. The cell suspension was transferred into a 1.5 mL EP tube, and centrifuged at 1600 rpm for 5 min. The supernatant was removed. 100 μL of 1×PBS was used in each tube to resuspend the cells respectively. Then 1 μL of a viability dye was added. The mixture was incubated at room temperature in the dark for 15 min after being mixed well. At the same time, the spleen was taken out with a tweezer, placed into a sterile petri dish containing 5 mL of the RPMI 1640 medium, and positioned on a 200-mesh cell screen. The spleen was gently ground with the needle core of a 10 mL syringe. A small amount of the RPMI 1640 medium was aspirated to rinse the cell screen. The spleen cell suspension was rinsed into a 15 mL centrifuge tube, and centrifuged at 1600 rpm for 5 min to collect cells. The cells were washed once with 1×PBS. 3 mL of an erythrocyte lysate (diluted with ddH₂O) was added to each tube to lyse for 5 min. After lysing, 10 mL of 1×PBS was added to each tube to stop lysing. The mixture was centrifuged at 1600 rpm for 5 min. 1 mL of 1×PBS was added to each 15 mL centrifuge tube. The mixture was transferred to a 1.5 mL EP tube, and centrifuged at 1600 rpm for 5 min. 1 mL of 1×PBS was added to each tube of the stained tumor cells and lysed spleen cells respectively to wash once. The mixture was centrifuged at 1600 rpm for 5 min, and the supernatant was removed. 100 μL of 1×PBS was used to resuspend the tumor cells and spleen cells respectively. 1 μL of the FcX™ antibody was then added to each tube. The mixture was incubated at room temperature in the dark for 10 min. After the incubation, 1 μL of each of the CD45, CD11b and Gr1 antibodies was added to each tube sequentially. The mixture was incubated at 4° C. in the dark for 30 min, and washed once with 1×PBS after the incubation. The supernatant was discarded. 300 μL of 1×PBS was used to resuspend the cells. The mixture was filtered through a filter membrane into a flow tube, and detected on a machine.

As shown in Table 25, in the H22 subcutaneous mouse xenograft models , after 2 weeks of continuous administration, arglabin and Compound 23 both have inhibitory effect on tumor growth.

TABLE 25
Anti-tumor effect of Compound 23 in H22 mouse xenograft models

| | | Number of | | | |
|---|---|---|---|---|---|
| | Dose | animals | Tumor weight | | Tumor growth |
| Group | (mg/kg/d) | start/end | (g) | | inhibition (%) |
| solvent | — | 6/6 | 8.56 | ± 1.72 | — |
| control | | | | | |
| 23 | 8.43 | 6/6 | 4.88 | ± 0.87** | 42.92% |
| arglabin | 10 | 6/6 | 5.18 | ± 0.61** | 39.43% |

*p < 0.05; **p < 0.01; and ***p < 0.001 (compared with the solvent control).

As shown in Table 26, after the experiment, the proportions of MDSCs in the tumor tissue and spleen in the H22 mouse subcutaneous xenograft models were detected by flow cytometry. Compared with the solvent control group, the proportions of MDSCs in the tumor tissue and spleen in arglabin and Compound 23 administration groups are significantly reduced.

TABLE 26
Inhibitory effect of Compound 23 on MDSCs in H22 mouse xenograft
models

| | Dose | MDSCs in tumor | | MDSCs in Spleen | |
|---|---|---|---|---|---|
| Group | (mg/kg/d) | (%) | | (%) | |
| blank group | — | — | | 6.06 | ± 0.94 |
| solvent control | — | 52.9 | ± 5.11 | 48.99 | ± 9.30*** |
| 23 | 8.43 | 7.86 | ± 6.19*** | 23.10 | ± 5.35*** |
| arglabin | 10 | 27.0 | ± 7.93*** | 27.89 | ± 4.72** |

*p < 0.05; **p < 0.01; and ***p < 0.001 (compared with the solvent control).

As shown in Table 27, after the experiment, the proportions of T cells in the tumor tissue and spleen in the H22 mouse subcutaneous transplanted tumor model were detected by flow cytometry. Compared with the solvent control group, the proportions of T cells in the tumor tissue and spleen in arglabin and Compound 23 administration groups are significantly increased.

TABLE 27
Effect of Compound 23 on T cells in H22 mouse xenograft models

| | Dose | T Cells in tumor | | T Cells in Spleen | |
|---|---|---|---|---|---|
| Group | (mg/kg/d) | (%) | | (%) | |
| blank group | — | — | | 18.22 | ± 0.96 |
| solvent control | — | 5.17 | ± 4.94 | 22.30 | ± 3.81 |
| 23 | 8.43 | 29.44 | ± 6.07*** | 37.19 | ± 6.99** |
| arglabin | 10 | 17.72 | ± 6.12** | 27.02 | ± 4.07 |

*p < 0.05; **p < 0.01; and ***p < 0.001 (compared with the solvent control)

### Example 17: Anti-Tumor Effect of Compound 23 in CT26 Subcutaneous Mouse Xenograft Models

### Experimental Method

CT26 cells were cultured and amplified in vitro. Appropriate amount of the cells in logarithmic growth phase were resuspended in a suspension of a serum-free RPMI 1640 medium and Matrigel (1:1) to prepare a cell suspension in 5×10⁵/100 μL under sterile conditions. 100 μL of the cell suspension was subcutaneously inoculated into the armpits of the front left limbs of male C57BL/6 mice with a syringe. When the tumor volume grew to 50-70 mm³, animals with moderate tumor size were randomly divided into groups, with 5 animals in each group. The groups were the solvent control group, the arglabin (20 mg/kg/d) group and the 23 (16.86 mg/kg/d) group with the same molar dose as arglabin, respectively. The mice were administered intragastrically every day for 3 weeks. During the administration, the weights and tumor diameters of the mice were measured every day. After the experiment, the mice were sacrificed by cervical dislocation, and the tumors were taken and weighed. The formula for calculating a tumor volume (TV) is: TV=½×a×b2, where a represents the long diameter of a tumor; b represents the short diameter of a tumor.

### Experimental Result

As shown in Table 28, in the Hepa1-6 mouse subcutaneous mouse xenograft models, after 2 weeks of continuous administration, similar to the positive compound arglabin, Compound 23 has significant inhibitory effect on tumor growth.

TABLE 28
Anti-tumor effect of Compound 23 in CT26 mouse xenograft models

| | | Number of | | | Tumor growth |
|---|---|---|---|---|---|
| | Dose | animals | Tumor weight | | inhibition |
| Group | (mg/kg/d) | start/end | (g) | | (%) |
| solvent control | — | 5/5 | 8.15 | ± 2.73 | — |
| 23 | 16.86 | 5/5 | 4.62 | ± 1.29* | 43.36% |
| arglabin | 20 | 5/5 | 5.31 | ± 0.51 | 34.94% |

*p < 0.05; **p < 0.01; and ***p < 0.001 (compared with the solvent control).

### Example 18: Safety Investigation of Compound 23

### Experimental Reagent

Compound 23 (purity >98%) was prepared according to the above Example 2; Sodium carboxyl methyl cellulose (CMC-Na), Sinopharm Chemical Reagent Co. Ltd.; and the other reagents are all analytically pure.

### Experimental Method

### Grouping and Administration of Mice

Female and male C57BL/6 mice, weighing 18-22 g, were randomly divided into the following three groups according to their weights to investigate the toxicity effect of Compound 23 on mice: the Normal group and Compound 23 (500 and 100 mg/kg) groups, with 10 mice in each group, and half of them were female and the other half were male. Compound 23 was administered intragastrically once a day for 30 days, with the administration volume of 0.1 mL/10 g, while the Normal group was administered the same volume of a solvent (normal saline).

### Specimen Collection

After the last administration, blood was collected from the venous plexus of fundus oculi, stood at room temperature for 2 h, and centrifuged at 3500 rpm and 4° C. for 15-20 min. The upper serum was aspirated, aliquoted, and frozen at −80° C. for subsequent use. After chloral hydrate anesthesia, the mice were sacrificed by the cervical dislocation method, and the abdominal skins of the mice were carefully cut open, and fixed with pins. Then the abdominal cavity and chest cavity of the mice were cut open. The livers and kidneys of the mice were taken out with tweezers, washed repeatedly with a pre-cooled PBS solution. Then small pieces of tissues were cut off and fixed in 10% neutral formalin solution (4% formaldehyde solution), and the remaining tissues were placed at −80° C. to be frozen for subsequent use.

### Experimental Result

### Effect on Body Weight of Mice

As shown in Table 29, during the duration of the experiment, the body weights of the experimental mice fluctuate within the normal range. Compared with the Normal group, 23 (500 and 1000 mg/kg) has no obvious effect on the body weights of female and male C57BL/6 mice (P>0.05).

TABLE 29
Effect of Compound 23 on body weight of mice

| | female | | | male | | |
|---|---|---|---|---|---|---|
| | | 23 | 23 | | 23 | 23 |
| Group | Normal | (500 mg/kg) | (1000 mg/kg) | Normal | (500 mg/kg) | (1000 mg/kg) |
| Day 1 | 100.00 ± 0.00 | 100.00 ± 0.00 | 100.00 ± 0.00 | 100.00 ± 0.00 | 100.00 ± 0.00 | 100.00 ± 0.00 |
| Day 5 | 98.37 ± 1.00 | 100.41 ± 0.43 | 101.96 ± 0.55 | 99.81 ± 2.21 | 100.31 ± 0.89 | 101.48 ± 1.30 |
| Day 10 | 98.61 ± 0.41 | 99.01 ± 1.34 | 101.80 ± 2.02 | 100.45 ± 0.91 | 101.51 ± 4.00 | 101.22 ± 0.84 |
| Day 15 | 101.72 ± 2.05 | 100.03 ± 0.95 | 99.77 ± 2.60 | 100.59 ± 1.24 | 99.27 ± 2.11 | 100.47 ± 1.28 |
| Day 20 | 98.10 ± 1.17 | 100.52 ± 0.74 | 101.04 ± 2.13 | 100.86 ± 0.67 | 101.29 ± 0.40 | 100.15 ± 2.76 |
| Day 25 | 100.21 ± 1.81 | 99.81 ± 1.72 | 99.06 ± 3.56 | 100.39 ± 0.74 | 98.93 ± 0.60 | 98.35 ± 1.14 |
| Day 30 | 100.12 ± 1.00 | 99.79 ± 2.55 | 100.67 ± 0.91 | 100.15 ± 0.92 | 100.10 ± 1.08 | 100.71 ± 0.53 |

### Effect on Activity State of Mice

Compared with the Normal group, the activities of the mice in the 23 (500 and 1000 mg/kg) groups are relatively reduced and their mental states are not good after each administration, but they can return to normal after 1-2 hours; and after 30 days of continuous observation, the hair of the mice is bright, and compared with the Normal group, there is no significant change in behavior activity and mental state, etc.

### Effect on Survival Ratio of Mice

As shown in Table 30, 23 (500 and 1000 mg/kg) has no obvious effect on survival ratio of female mice after 30 days of continuous administration. After administering continuously 23 (500 and 1000 mg/kg) for 30 days, none of the mice die, and the compound has no obvious effect on survival ratio of male mice.

TABLE 30
Effect of Compound 23 on survival ratio of mice

| | female | | | male | | |
|---|---|---|---|---|---|---|
| | | 23 | 23 | | 23 | 23 |
| | | (500 | (1000 | | (500 | (1000 |
| Group | Normal | mg/kg) | mg/kg) | Normal | mg/kg) | mg/kg) |
| survival ratio | 100% | 100% | 100% | 100% | 100% | 90.00% |

What is claimed is:

1. Guaianolide sesquiterpene lactone derivatives of general formula I, or a pharmaceutically acceptable salt thereof, wherein 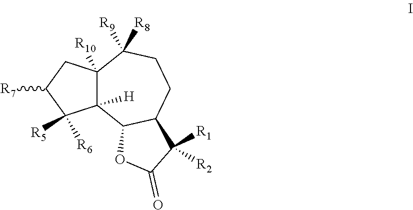
R¹ and R² together form a double bond; or R¹ is hydrogen or deuterium, R² is 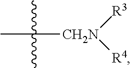
wherein R³ and R⁴ are C1-C6 alkyl groups respectively, or R³, R⁴ and a N atom form a 5-6 membered ring structure, on which the ring structure is selected from pyrrole, piperidine, piperazine, and morpholine;
R⁵ is methyl; and R⁶ is a hydroxyl, C1-C6 alkoxy, C1-C6 ester group, halogen or forms a double bond with an adjacent carbon atom;
R⁷ is hydrogen or hydroxyl;
R⁸ is methyl; and R⁹ is connected with R¹⁰ to form cyclopropane.

2. The guaianolide sesquiterpene lactone derivatives or a pharmaceutically acceptable salt thereof according to claim 1, which is selected from the following compounds:

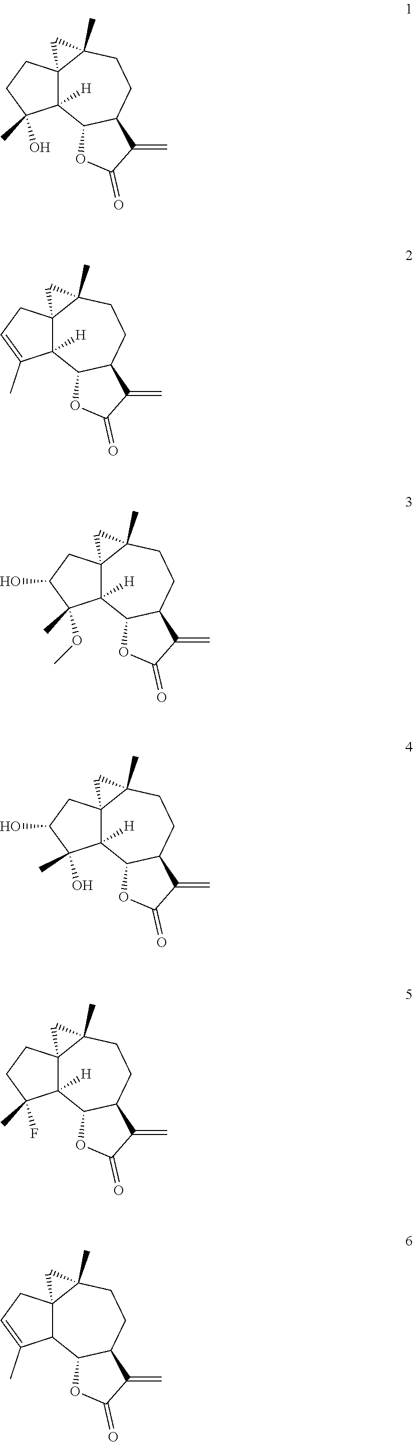

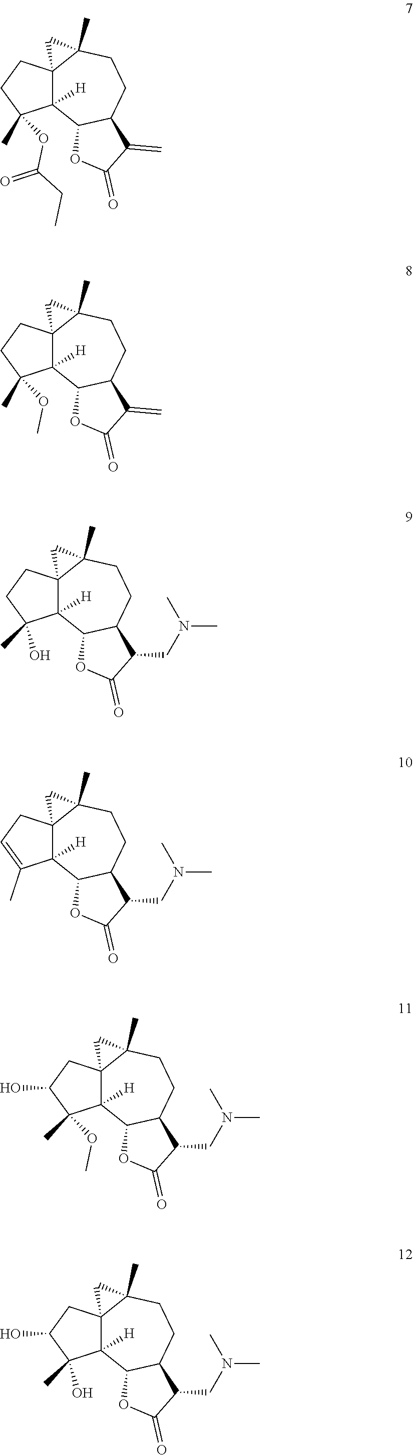

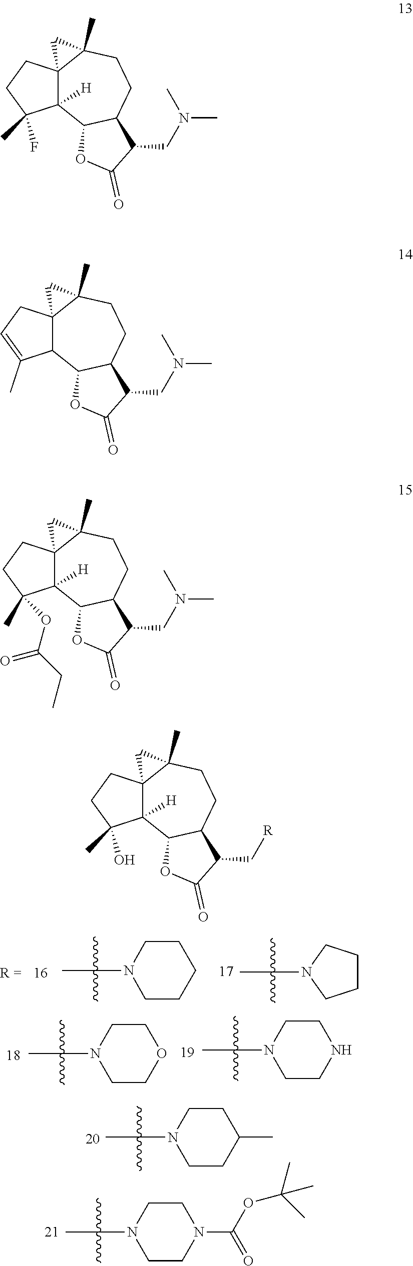

3. The guaianolide sesquiterpene lactone derivatives or a pharmaceutically acceptable salt thereof according to claim 1, wherein the pharmaceutically acceptable salt is selected from a hydrochloride, a sulfate, a phosphate, a maleate, a fumarate and a citrate.

4. The guaianolide sesquiterpene lactone derivatives or a pharmaceutically acceptable salt thereof according to claim 3, wherein the pharmaceutically acceptable salt is selected from:

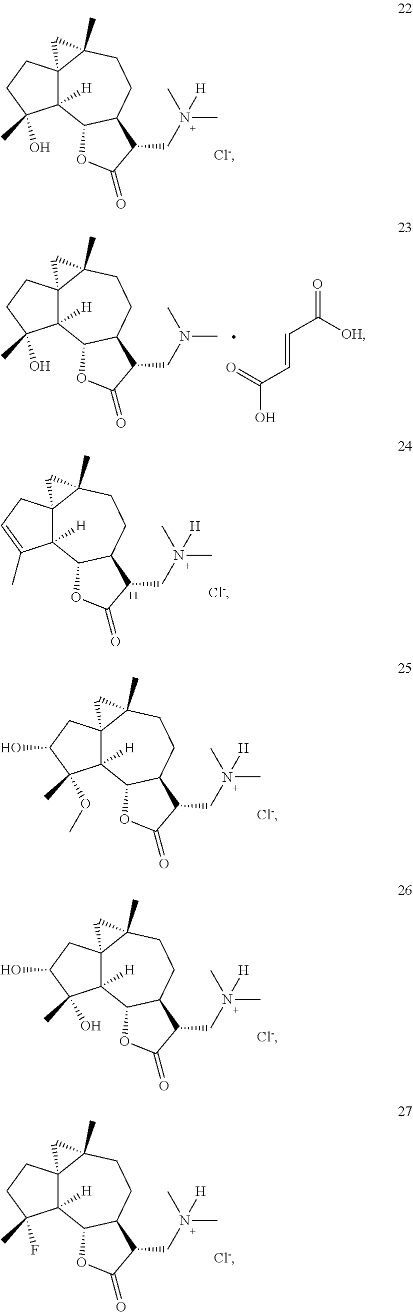

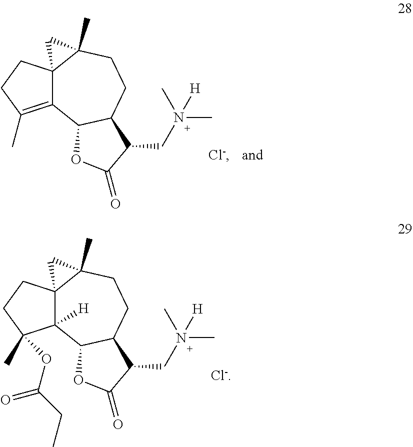

5. Use of the guaianolide sesquiterpene lactone derivatives or a pharmaceutically acceptable salt thereof according to claim 1 in preparation of a medicine for treating NLRP3 inflammasome-associated diseases.

6. The use according to claim 5, wherein the NLRP3 inflammasome-associated diseases are selected from immune diseases, autoimmune diseases, malignant tumor, skin diseases, cardiovascular diseases, liver-associated diseases, kidney system-associated diseases, gastrointestinal tract-associated diseases, central nervous system diseases, metabolic diseases, endocrine-associated diseases, respiratory diseases, lymphatic system diseases, inflammation, infectious diseases, ocular diseases, psychological disorders and pain.

7. The use according to claim 5, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

8. A pharmaceutical composition, comprising the guaianolide sesquiterpene lactone derivatives or a pharmaceutically acceptable salt thereof according to claim 1, and a pharmaceutically acceptable carrier.

9. Use of the pharmaceutical composition according to claim 8 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

10. Use of the guaianolide sesquiterpene lactone derivatives or a pharmaceutically acceptable salt thereof according to claim 2 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

11. Use of the guaianolide sesquiterpene lactone derivatives or a pharmaceutically acceptable salt thereof according to claim 3 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

12. Use of the guaianolide sesquiterpene lactone derivatives or a pharmaceutically acceptable salt thereof according to claim 4 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

13. A pharmaceutical composition, comprising the guaianolide sesquiterpene lactone derivatives or a pharmaceutically acceptable salt thereof according to claim 2, and a pharmaceutically acceptable carrier.

14. Use of the pharmaceutical composition according to claim 13 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers: hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

15. A pharmaceutical composition, comprising the guaianolide sesquiterpene lactone derivatives or a pharmaceutically acceptable salt thereof according to claim 3, and a pharmaceutically acceptable carrier.

16. Use of the pharmaceutical composition according to claim 15 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases:
chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers:
hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.

17. A pharmaceutical composition, comprising the guaianolide sesquiterpene lactone derivatives or a pharmaceutically acceptable salt thereof according to claim 4, and a pharmaceutically acceptable carrier.

18. Use of the pharmaceutical composition according to claim 17 in preparation of a medicine for treating NLRP3 inflammasome-associated diseases, wherein the NLRP3 inflammasome-associated diseases are selected from: (1) Cryopyrin-associated periodic syndromes (CAPS): Muckle-Wells syndromes (MWS), familial cold autoinflammatory syndromes (FCAS) and neonatal-onset multisystem inflammatory diseases (NOMID); (2) autoinflammatory diseases: familial Mediterranean fever (FMF), TNF receptor-associated periodic syndromes (TRAPS), mevalonate kinase deficiency (MKD), hyperimmunoglobulin D and periodic fever syndromes (HIDS), deficiency of interleukin-1 receptor (DIRA), Majeed syndromes, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), and sideroblastic anemia with B-cell immunodeficiency, periodic fever and developmental delay (SIFD); (3) Sweet's syndromes: chronic nonbacterial osteomyelitis (CNO), chronic recurrent multifocal osteomyelitis (CRMO), and synovitis, acne, pustulosis, hyperostosis, and osteitis syndromes (SAPHO); (4) autoimmune diseases: multiple sclerosis MS, type 1 diabetes, psoriasis, rheumatoid arthritis, Behcet's diseases, Sjogren's syndromes, and Schnitzler syndromes; (5) respiratory system diseases: acute lung injury, chronic obstructive pulmonary disease (COPD), steroid-resistant asthma, asbestosis, silicosis and cystic fibrosis; (6) central nervous system diseases: Parkinson's diseases, Alzheimer's diseases, motor neuron diseases, Huntington's diseases, cerebral malaria and brain injury from pneumococcal meningitis; (7) metabolic diseases: type 2 diabetes, atherosclerosis, obesity, gout, acute gouty arthritis and pseudogout; (8) ocular diseases: ocular epithelium diseases, age-related macular degeneration (AMD), corneal infection, uveitis and xerophthalmia; (9) kidney-associated diseases: chronic kidney diseases, oxalate nephropathy and diabetic nephropathy; (10) liver-associated diseases: hepatitis, non-alcoholic steatohepatitis and alcoholic liver diseases; (11) skin-associated inflammatory responses: contact hypersensitivity and sunburn; (12) joint-associated inflammatory responses: osteoarthrosis, systemic juvenile idiopathic arthritis, adult Still's diseases, and relapsing polychondritis; (13) viral infections: Dengue virus and Zika virus, influenza, and HIV virus; (14) hidradenitis suppurativa (HS) and other cyst-causing skin diseases; (15) cancers:
hepatocellular carcinoma, colon cancer, lymphoma, lung cancer, pancreatic cancer, gastric cancer, myelodysplastic syndromes, abdominal aortic aneurysm and leukemia; and (16) polymyositis, ulcerative colitis, Crohn's disease, pericarditis, worm infections, sepsis caused by bacterial and viral infections, wound healing, depression, stroke, myocardial infarction, hypertension, Dressler's syndromes, and ischemia-reperfusion injury.
